SEQUENCE LISTING (14 sequences)

SEQ ID NO: 1 - DNA, 25 bp, Artificial Sequence
  hCD10 forward primer
ttgttggcactgatgataagaattc

SEQ ID NO: 2 - DNA, 29 bp, Artificial Seqeunce
  hCD10 reverse primer
tccagtgcattcatagtaatctctagaag

SEQ ID NO: 3 - DNA, 25 bp, Artificial Seqeunce
  hCD200 forward primer
tgtgaccgactttaagcaaaccgtc

SEQ ID NO: 4 - DNA, 23 bp, Artificial Seqeunce
  hCD200 reverse primer
ttagggctctcggtcctgattcc

SEQ ID NO: 5 - DNA, 20 bp, Artificial Seqeunce
  mCD10 forward primer
gaactttgcccaggtgtggt

SEQ ID NO: 6 - DNA, 20 bp, Artificial Seqeunce
  mCD10 reverse primer
gcggcaatgaaaggcatctg

SEQ ID NO: 7 - DNA, 21 bp, Artificial Seqeunce
  mCD200 forward primer
gagcacagctcaagtggaagt

SEQ ID NO: 8 - DNA, 20 bp, Artificial Seqeunce
  mCD200 reverse primer
gttttctgggctcacggctt

SEQ ID NO: 9 - DNA, 24 bp, Artificial Seqeunce
  hPPARG forward primer
gacaggaaagacaacagacaaatc

SEQ ID NO: 10 - DNA, 21 bp, Artificial Seqeunce
  hPPARG reverse primer
ggggtgatgtgtttgaacttg

SEQ ID NO: 11 - DNA, 27 bp, Artificial Seqeunce
  haP2 forward primer
cctttaaaaatactgagatttccttca

SEQ ID NO: 12 - DNA, 20 bp, Artificial Seqeunce
  haP2 reverse primer
ggacacccccatctaaggtt

SEQ ID NO: 13 - DNA, 23 bp, Artificial Seqeunce
  hGAPDH/ mGapdh forward primer
caaggtcatccatgacaactttg

SEQ ID NO: 14 - DNA, 20 bp, Artificial Seqeunce
  hGAPDH/ mGapdh reverse primer
ggccatccacagtcttctgg

(19) United States
(12) Patent Application Publication
(10) Pub. No.: US 2020/0319161 A1
(43) Pub. Date: Oct. 8, 2020

(54) METHOD OF IDENTIFYING ADIPOSE STEM CELLS

(71) Applicant: AGENCY FOR SCIENCE, TECHNOLOGY AND RESEARCH, Singapore (SG)

(72) Inventors: Shigeki SUGII, Singapore (SG); Wee Kiat ONG, Singapore (SG); Weiping HAN, Singapore (SG)

(21) Appl. No.: 16/869,501

(22) Filed: May 7, 2020

Related U.S. Application Data
(62) Division of application No. 16/100,152, filed on Aug. 9, 2018, now abandoned.
Application No. 16/100,152 is a division of application No. 14/907,269, filed on Jan. 22, 2016, now abandoned.

(30) Foreign Application Priority Data
Aug. 1, 2013 (SG) 201305877-1

(51) Int. Cl. G01N 33/50; G01N 33/68; C12N 5/0775

(52) U.S. Cl. CPC G01N 33/5005; G01N 33/6872; G01N 2500/10; G01N 2333/70596; C12N 2501/115; C12N 5/0667

(57) ABSTRACT

Disclosed is a method of identifying the origin of adipose-derived stem cells, comprising detecting at least one cell-surface marker selected from a group consisting of CD 10, CD141, CD 142 and CD200. Also disclosed are methods of determining adipogenic capability of a cell capable of adipogenesis by determining the presence of CD 10 or CD200.

### CROSS-REFERENCE TO RELATED APPLICATIONS

This application claims the benefit of priority of Singapore provisional application No. 201305877-1, filed Aug. 1, 2013, the contents of it being hereby incorporated by reference in its entirety for all purposes.

### FIELD OF THE INVENTION

The present invention relates generally to the field of molecular biology. In particular, the present invention relates to and the use of molecular markers for the identification of the origin and for the selection of adipose stem cells.

### BACKGROUND OF THE INVENTION

Adipose tissue, such as white adipose tissue (WAT) has been increasingly appreciated as an alternative source of mesenchymal stem cells traditionally isolated from the bone marrow. For example, subcutaneous white adipose tissue can be isolated by minimally invasive liposuction procedure. Additionally, adipose-derived stem cells are relatively abundant in the adipose tissue where as much as 1% of human adipose cells are adipose-derived stem cells, compared to only 0.001-0.002% mesenchymal stem cells in the bone marrow. The differentiation capacity, immunobiological properties and secretome of adipose-derived stem cells offer tremendous therapeutic potential in regenerative medicine.

Increasing evidence suggests that stem cells, derived from adipose tissue of different depot origins, are distinct populations of cells that differ in their inherent properties, e.g. their differentiation ability in response to in vitro stimuli. The functional difference of adipose-derived stem cells of different origins, together with regional variation in cellular interaction, circulation, innervations and anatomic constraints between different depots of adipose tissue, are thought to be the underlying factors contributing to pathophysiological variation of adipose tissue depots in relation to metabolic homeostasis. For example, the subcutaneous depot physiologically stores excess lipids, thus preventing their deposition into other organs. Accumulation of fat in the visceral depot, on the other hand, leads to a pathological metabolic profile because of dysfunction in lipid storage. Differences in these properties are, at least partly, cell autonomous and recapitulated in vitro in stem cells derived and isolated from these depots.

Further characterisation of adipose-derived stem cells is necessary to understand their pathophysiological roles in metabolism and therapeutic relevance in regenerative medicine. Thus, it is an object of the present invention to identify methods that help to differentiate adipose-derived stem cells from different origins.

### SUMMARY

In a first aspect, the present invention refers to a method of identifying the origin of adipose-derived stem cells, comprising detecting at least one cell-surface marker selected from a group consisting of CD10, CD141, CD142 and CD200.

In a second aspect, the present invention refers to a method of determining adipogenic capability comprising determining the presence of CD10 in a cell capable of adipogenesis.

In a third aspect, the present invention refers to a method of determining adipogenic capability comprising determining the presence of CD200 in a cell capable of adipogenesis.

In a fourth aspect, the present invention refers to a method of using cell surface markers, as defined herein, wherein the cell surface markers are used in a high-throughput screening assay for drugs or compounds that would enhance adipogenesis.

### BRIEF DESCRIPTION OF THE DRAWINGS

The invention will be better understood with reference to the detailed description when considered in conjunction with the non-limiting examples and the accompanying drawings, in which:

FIG. 1 shows images representing the various stages and the validation used during high content screening of adipose-derived stem cells for potential depot-specific cell surface markers. (A) shows a graphic representation of the strategy applied for screening and identification of cell surface markers. (B) shows representative fluorescence images of subject S1-derived subcutaneous adipose-derived stem cells and visceral adipose-derived stem cells immunostained with conventional mesenchymal stem cell markers (CD73, CD90 and CD105 in red). Hoechst 33342 was used to stain nuclei of all live cells in blue. Bone-marrow-derived mesenchymal stem cells and human foreskin fibroblasts (HFF-1) were used as controls. (C) shows further representative fluorescence images suggesting depot-specific expression of CD10 and CD141 in subcutaneous-derived adipose stem cells (subject S1 and commercial source) and that of CD142 and CD200 in visceral adipose-derived stem cells (subject S1). The cells were stained with Alexa Fluor 647-conjugated secondary antibody, following primary staining with antibodies from BD Lyoplate™ Human Cell Surface Marker Screening Panel, and the nuclei were counterstained with Hoechst 33342. The fluorescence images for all other markers with at least one positive signal from the Lyoplate screening are available in FIGS. 11 to 68.

FIG. 2 depicts the mRNA and protein expression profiles of CD10 and CD200 in subcutaneous and visceral adipose-derived stem cells across subjects. (A) shows line graphs representing qPCR data showing the relative expression level of CD10 and CD200 between subcutaneous adipose-derived stem cells (expression level normalized as 1) and visceral adipose-derived stem cells across subjects S1 to S6. (B) shows images of a Western blot analysis showing the protein expression of CD10 between subcutaneous adipose-derived stem cells and visceral adipose-derived stem cells across subjects S1 to S3 and in commercial subcutaneous adipose-derived stem cells. The protein expression of CD200 was not shown due to unavailability of an antibody with satisfactory quality for Western blotting.

FIG. 3 shows histograms representing the results of a flow cytometry analysis of subcutaneous and visceral adipose-derived stem cells populations. The histograms shown in (A) show that both the subcutaneous and visceral adipose-derived stem cells of all subjects expressed the conventional mesenchymal stem cell markers, as represented by subject S1. (B) depicts histograms showing CD10-expressing cell populations that were almost exclusively found in the subcutaneous adipose-derived stem cells across subjects S1 to S12. (C) further shows histograms showing that CD200-expressing cell populations were predominantly found in the visceral adipose-derived stem cells across subjects S1 to S12.

FIG. 4 shows histograms illustrating the correlation of CD10 and CD200 expression levels and adipogenesis. The data depicted in (A) shows that subcutaneous adipose-derived stem cells differentiated better than their visceral adipose-derived stem cells counterparts into adipocytes in response to standard adipogenic stimulation. Adipogenesis of adipose-derived stem cells was induced using the standard adipogenic cocktail including indomethacine for D0-D4, followed by maintenance medium with insulin alone (D4-D12). The mRNA expression level of CD10 positively correlated with induction of adipogenesis, as determined by qPCR. On the other hand, a negative correlation was observed for CD200 (data represented by S6). (B) shows column histograms and Western blot images, showing increased CD10 protein expression in response to adipogenic stimulation. The densitometry reading of CD10, measured by ImageJ software, was normalised against β-actin as a loading control (represented by commercial subcutaneous adipose-derived stem cells). (C) shows data illustrating that CD10^{hi} cells sorted from subcutaneous adipose-derived stem cells differentiated better than their CD10^{lo} counterparts. Representative data is shown for subject S4, with similar results obtained in other subjects. (D) shows histograms and images of CD200^{lo} cells sorted from visceral adipose-derived stem cells showed that these differentiated better than the CD200^{hi} counterparts. Representative data is shown for subject S2, with similar results seen in other cells. Statistical significance was assessed by using Student's t-test.

FIG. 5 shows representative images showing higher adipogenic potential of subcutaneous adipose-derived stem cells in comparison with respective visceral adipose-derived stem cells isolated from 4 subjects. Adipose-derived stem cells were subjected to standard adipogenic stimuli, and stained with Oil Red O.

FIG. 6 shows a fluorescence image montage highlighting the consistent depot-dependant expression of CD10 and CD200 across subjects S1 to S3 in the follow-up high content screening. In contrast, other cell surface markers represented by CD141 and CD142 were not consistent across individuals.

FIG. 7 shows column graphs of qPCR data illustrating the relative expression level of CD10 and CD200 between subcutaneous adipose-derived stem cells from inguinal region (expression level defined as 1) and visceral adipose-derived stem cells from epididymal (EP) and mesenteric (MS) regions. These adipose-derived stem cells were isolated from two pools of five mice, fed with normal chow (N) or high-fed diet (H).

FIG. 8 shows line graphs of qPCR data showing the mRNA expression level of adipogenic markers (PPARγ and aP2) during the course of adipogenesis. Adipogenesis of adipose-derived stem cells was induced using the standard adipogenic cocktail including indomethacine for day 0 to day 4 (D0-D4), followed by maintenance medium with insulin alone (day 4 to day 12). The expression level of PPARγ and aP2 positively correlated with CD10 expression but negatively correlated with that of CD200 (see FIG. 4A).

FIG. 9 shows histograms illustrating the flow cytometry sorting of (A) subcutaneous adipose-derived stem cells into CD10^{hi} and CD10^{lo} populations (representative data shown for subject S4) and (B) visceral adipose-derived stem cells into CD200^{hi} and CD200^{lo} populations (representative data shown for subject S2).

FIG. 10 shows micrograph images illustrating the results of osteogenesis and chondrogenesis assays of isolated ASCs. Images show Alizarin Red S (osteoblast dye) and Alcian Blue (chondrocyte dye) staining of subcutaneous adipose-derived stem cells.

FIGS. 11 to 68 shows fluorescence images of 58 cell surface markers that had at least one positive signal in any of the five cell lines tested. The cells were stained with Alexa Fluor 647-conjugated secondary antibody, following primary staining with antibodies from BD Lyoplate™ Human Cell Surface Marker Screening Panel, and the nuclei were counterstained with Hoechst 33342. Two images were provided for each marker/cell line, with the left images showing the cell surface marker staining alone and the right images showing the cell surface marker staining together with the Hoechst staining. Average signal intensities of the individual cell surface markers were assessed by MultiWave Scoring using the MetaXpress software. The Plate Acquisition Summary is as follows: 80 ms for wavelength DAPI (for Hoechst 33342 staining) and 500 ms for wavelength Cy5 (for Alexa Fluor 647 staining). The image scaling was used for Cy5 (for Alexa Fluor 647 stained samples) with a minimum of 105 and a maximum of 200.

### DETAILED DESCRIPTION OF THE PRESENT INVENTION

Adipose-derived stem cells (ASCs) from the anatomically distinct adipose tissue, such as subcutaneous and visceral depots of white adipose tissues (WATs), differ in their inherent properties. However, little is known about the molecular identity and definitive markers of adipose-derived stem cells from these depots. In view of this, there is a need to develop a method for identifying the origin of adipose-derived stem cells, tracking and screening of depot-specific stem cell populations. To this end, adipose-derived stem cells from exemplary sources, such as subcutaneous fat (SC-ASCs) and visceral fat (VS-ASCs) of omental region, were isolated and studied. High content image screening of over 240 cell surface markers identified several potential depot-specific markers of adipose-derived stem cells. Hence, the present disclosure provides a method of identifying the origin of adipose-derived stem cells by the detection of cell-surface markers.

As used herein, the term “identifying” refers to the ability to define or name a cell, cell-type or origin of a cell based on defined indicators or assay results, e.g. a cell expressing a certain marker would be deemed to have originated from an origin known to produce or result in cells having said marker. Indicators and methods that can be used for this purpose can be protein expression, mRNA, RNA, DNA, RNAi based identification, genomic analysis, morphological analysis and reference images, as well as cell-surface markers, for example cluster of differentiation (CDs), and immunophenotyping. The identification may also be made in relative terms, denoting that a higher expression of a given indicator may point toward one cell identity, while a lower expression of the same indicator may point toward a different cell identity, with the relative basis being defined in comparison to a normal cell or the cell type to be achieved.

The term “origin”, as used herein, refers to the anatomical location within a mammalian body and may also refer to the tissue type from which the analysed cell was isolated. Examples of anatomical and tissue-specific origins may be cardiac, cutaneous, subcutaneous, visceral, dermal, sub-dermal, hepatic, pancreatic, bronchial, muscular, skeletal, cardiovascular, lymphatic, endocrinal, immunological, nervous, respiratory, reproductive and cerebral. In one example, the method is as disclosed herein, wherein the origin of the identified cell is subcutaneous. In another example, the origin of the identified cell is visceral.

As used herein, the term “adipose” or “adipose tissue” refers to connective tissue in which fat is stored and in which cells are distended by droplets of fat. This connective tissue, while comprising mostly of adipocytes, may also contain the stromal vascular fraction (SVF) of cells, including pre-adipocytes, fibroblasts, vascular endothelial cells and a variety of immune cells (i.e. adipose tissue macrophages (ATMs)). Various types of adipose tissue have been found in humans and most mammals, namely white adipose tissue (WAT) and brown adipose tissue (BAT). One example of a specific subgroup of white adipose tissue is white adipose tissue with inducible thermogenic capabilities.

As used herein the term “adipose-derived stem cells (ASCs),” refers to stem cells that have been derived or isolated from adipose tissue. By the convention of International Society for Cellular Therapy (ISCT), mesenchymal stem cells from various sources, including adipose-derived stem cells, are defined as being (i) plastic-adherent in the standard cell culture condition, (ii) multipotent, i.e., able to differentiate into osteoblasts, adipocytes and chondrocytes in vitro and (iii) positive for CD73, CD90 and CD105, and negative for CD11 b or CD14, CD19 or CD79a, CD34, CD45 and HLA-DR in their cell surface immunophenotype. As described herein, the adipose tissue referred to can be white or brown adipose tissue, the main difference between the two being in their function, with white adipose tissue being used as energy storage, and the primary function of brown adipose tissue being the generation of body heat.

As used herein and in the art, the phrases “precursor cell,” “progenitor cell,” and “stem cell” may be used interchangeably and refer to either a pluripotent or lineage-uncommitted progenitor cell, which is potentially capable of an unlimited number of mitotic divisions to either renew itself or to produce progeny cells, which will differentiate into the desired cell type. In contrast to pluripotent stem cells, lineage-committed progenitor cells are generally considered to be incapable of giving rise to numerous cell types that phenotypically differ from each other. In mammals, there are two broad types of stem cells known: embryonic stem cells, which are isolated from the inner cell mass of blastocysts, and adult stem cells, which are found in various tissues. In mature organisms, stem cells and progenitor cells act as a repair system for the body, replenishing adult tissue. In a developing embryo, stem cells can differentiate into all the specialized cells, namely ectoderm, endoderm and mesoderm, but also maintain the normal turnover of regenerative organs, such as blood, skin, or intestinal tissues.

As used herein, the prefix “CD” in the context of, e.g. CD10 stands for “cluster of differentiation” and is also interchangeably used in the art with the term “cell surface marker”. In this example, CD10 refers to the presence of the cluster of differentiation molecule number 10 on cells in the human body. In some instances, the name of the cluster of differentiation may also refer to the gene encoding said molecule. This term “CD” may be additionally annotated using the term “+”, which denotes the presence of the cluster, whereas “−” denotes the absence thereof.

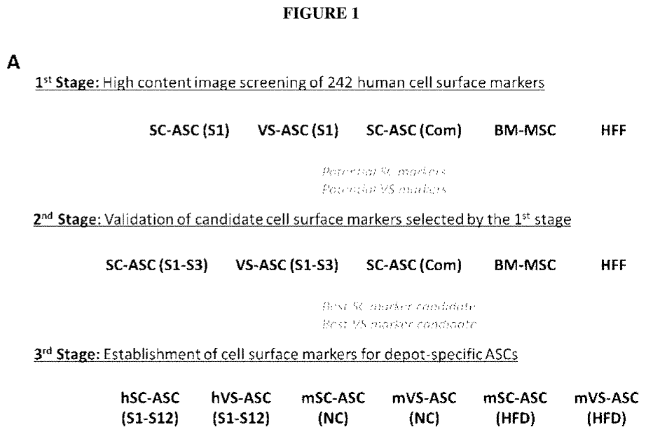

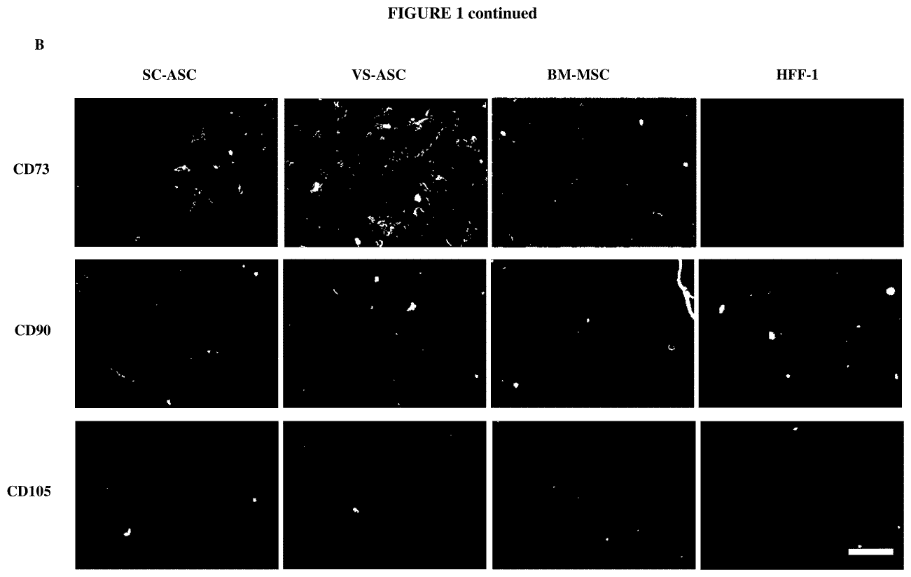

Here, an approach was taken to identify depot-specific adipose-derived stem cell markers, whereby high content screening assay of 242 human cell surface markers was performed on five cell lines. Additional experiments were performed for further verification of selected candidate markers in those five cell lines. Examples of these selected candidate markers included CD10 and CD141 as potential subcutaneous adipose-derived stem cells markers, whereas CD142 and CD200 were selected as potential visceral adipose-derived stem cells (FIG. 1C).

As a result, there is described herein a method of identifying the origin of adipose-derived stem cells, the method comprising detecting at least one or two or three or four cell-surface makers selected from a group consisting of CD10, CD141, CD142 and CD200.

As used herein, CD10, also known as neprilysin, neutral endopeptidase, enkephalinase and common acute lymphoblastic leukemia antigen—CALLA, refers to a cell surface metallopeptidase that inactivates a number of signalling substrates. Further, CD10 is known in the art as a cell surface marker of mammary stem cells and that sorting by CD10 enriches sphere-forming stem/progenitor populations.

As used herein, the term “CD141”, also known as Thrombomodulin™ or BDCA-3, refers to an integral membrane protein generally expressed on the surface of endothelial cells and serves as a cofactor for thrombin. Its main function is the reduction of blood coagulation via the conversion of thrombin to an anticoagulant enzyme from a pro-coagulant enzyme.

As used herein, the term “CD142” refers to a protein, which is also known as tissue factor, platelet tissue factor, coagulation factor III and thromboplastin. CD142 is generally present in sub-endothelial tissue and leukocytes, which are necessary for initiating thrombin formation from its zymogen prothrombin, however is known to absent from cells which are in constant contact with plasma.

As used herein, the term “CD200”, also known as OX-2 membrane glycoprotein (OX-2), refers to a type I membrane protein and is known to be commonly expressed on cells originating from the hematopoietic cells, on B-cells, activated T-cells, endothelial neuronal cells and cells of the reproductive organs (ovaries and placental trophoblasts). CD200 is also a member of the immunoglobulin superfamily of proteins, whose biological function is unclear but implicated in multiple immuno-regulatory activities of myeloid and other immune cell types.

Further studies were performed to study the expression of selected cell surface marker candidates across various subjects. It was shown that CD10 was predominantly expressed in subcutaneous adipose-derived stem cells and that the expression of CD200 in visceral adipose-derived stem cells was consistent.

Hence, there is disclosed a method as described herein, wherein the presence of CD10 and/or CD141 is/are indicative for the presence of adipose-derived stem cells from subcutaneous depots (SC). Further, there is also described a method, as defined herein, wherein the presence of CD200 and/or CD142 is/are indicative for the presence of adipose-derived stem cells from visceral depots (VC).

It was also found that the expression for CD10 and CD200 was more consistent than the expression of CD141 and CD142 throughout all subjects, with these variations also being found in other cell surface markers. Therefore, CD10 and CD200 were selected for further studies.

Hence, in one example, there is disclosed a method is as described herein, wherein the cell-surface maker is CD10. In another example, the cell-surface marker is CD200.

The expression analysis of CD10 and CD200 showed that the mRNA expression of CD10 was consistently higher in the subcutaneous adipose-derived stem cells relative to visceral adipose-derived stem cells, whereas that of CD200 was consistently lower in the subcutaneous adipose-derived stem cells relative to visceral adipose-derived stem cells across various subjects, as well as in mice. This result suggests that the depot specificity of CD10 and CD200 also holds true in mouse species. The predominant expression of CD10 by subcutaneous adipose-derived stem cells and the higher expression of CD200 by visceral adipose-derived stem cells were confirmed. These validation results further established CD10 as a subcutaneous adipose-derived stem cells-specific marker and CD200 as a visceral adipose-derived stem cells-enriched marker. Hence, the presence of at least one of these cell-surface markers, that is at least one of the clusters of differentiations, is indicative of both the identity and the origin of the analysed cell. Similar results can be found for CD141 and CD142.

Hence, there is described herein is a method, as defined herein, wherein the adipose-derived stem cells are from visceral depots (VS) or from subcutaneous depots (SC). These depots may contain either white or brown adipose tissue. As such, in a further example, the visceral or subcutaneous depots are of white adipose tissue, white adipose tissue with thermogenic capabilities or brown adipose tissue. In another example, the visceral or subcutaneous depots are of white adipose tissue.

As used herein, the terms “visceral” and “subcutaneous” refer to specific anatomical locations from which the cell was isolated, with “subcutaneous” referring to the layer containing fat and connective tissue further housing larger blood vessels and nerves found beneath the dermis in an area known as the hypodermis, and “visceral” referring to the locations around internal organs.

As used herein, the term “depot” refers to any area of the body in which drugs or other substances, such as fat, are stored and from which they may be distributed. In the present disclosure, depot refers to the area within the mammalian body, in which an abundance of adipose tissue may be found.

As used herein, the terms “white adipose tissue (WAT)” “white adipose tissue with inducible thermogenic capabilities” and “brown adipose tissue (BAT)” refer to subtypes of adipose tissue in mammals. The main difference between them is their function, with white adipose tissue being used mainly as energy storage, and the primary function of brown adipose tissue being the generation of body heat. To this end and in contrast to white adipose tissue in which adipocytes contain a single lipid fat droplet, the adipocytes in brown adipose tissue contain numerous smaller fat droplets and a greater number of (iron-containing) mitochondria, resulting in its distinct brown colour. Brown adipose tissue also contains more capillaries than white adipose tissue, since it has a greater need for oxygen than most tissues.

It was also observed that the expression level of CD10 and CD200 during adipogenesis showed that subcutaneous adipose-derived stem cells differentiated better into mature adipocytes than visceral adipose-derived stem cells using the standard in vitro adipogenesis protocol. The mRNA expression level of CD10 increased after adipogenic stimuli. In contrast, the CD200 level decreased after adipogenesis is initiated. Further, the adipogenic potential of adipose-derived stem cell subpopulations expressing high/low level of CD10 or CD200 was analysed by sorting subcutaneous adipose-derived stem cells into two populations: CD10^{hi} and CD10^{lo} cells, and visceral adipose-derived stem cells into CD200^{hi} and CD200^{lo} cells. After subjecting these populations to the standard in vitro adipogenesis cocktail, the results indicated that CD10^{hi} cells sorted from subcutaneous adipose-derived stem cells differentiate significantly better than their CD10^{lo} counterparts, and that CD200^{lo} cells sorted from visceral adipose-derived stem cells were found to differentiate significantly better than the CD200^{hi} counterparts. Therefore, this data showed that CD10 is a prospective marker for high adipogenic potentials, whereas CD200 is a predictive marker for lower adipogenic capacities. These results are also indicative of the same for cell types that are generally capable of adipogenesis. Without being bound by theory, preliminary indications (data not shown) show that CD10 and CD200 may not only serve as depot-specific markers, but may also play general key roles in adipogenic pathways. In other words, any process or intervention resulting in a change in CD10 and/or CD200 expression may help to influence, and possibly improve, functional differentiation of any cells that have adipogenic capacity.

Disclosed herein is a method of determining adipogenic capability, comprising determining the presence of CD10 in a cell capable of adipogenesis. In one example, the method is as defined herein, wherein the adipogenic capability is identified by the determination of the concentration of CD10, wherein an increased concentration of CD10 (CD10^{hi}) is indicative of a cell capable of adipogenesis having a higher adipogenic capability compared to the average cell capable of adipogenesis. In another example, the cell capable of adipogenesis includes, but is not limited to adipose-derived stem cells, pre-adipocytes, adipose progenitor cells, pericytes, smooth muscle cells, muscle progenitor cells, mesenchymal stem cells, stromal cells, fibroblasts with adipogenic potentials, embryonic stem cells, induced pluripotent stem cells and combinations thereof.

Also disclosed herein is a method of determining adipogenic capability, comprising determining the presence of CD200 in a cell capable of adipogenesis. In one example, the method is as defined herein, wherein the adipogenic capability is identified by the determination of the concentration of CD200, wherein a reduced concentration of CD200 present)(CD200^{lo} is indicative of the cell capable of adipogenesis having an higher adipogenic capability compared to the average cell capable of adipogenesis. In another example, the cell capable of adipogenesis is an adipose-derived stem cell, as defined herein. In a further example, the method of determining adipogenic capability is as described herein, wherein the adipogenic capability is identified by the determination of the concentration of CD10, wherein a high concentration of CD10 (CD10^{hi}) is indicative of an adipose-derived stem cell from subcutaneous depots (SC) of white adipose tissue having an higher adipogenic capability compared to the average adipose-derived stem cell of subcutaneous depot origin. In another example, the adipogenic capability is identified by the determination of the concentration of CD200, wherein a low concentration of CD200 present)(CD200^{lo} is indicative of an adipose-derived stem cell from visceral depots (VS) of white adipose tissue having an higher adipogenic capability compared to the average adipose-derived stem cell of visceral depot origin.

As used herein, the term “determine” or grammatical variations thereof, refers to the act of to establishing or deciding (something), especially if the decision is based on evidence or facts. In the present example, the correlation between CD10/CD200 expression and the adipogenic ability of the various cell-types was correlated and stated using results of various scientific analyses.

As used herein, the term “adipogenic capacity” or “adipogenic potential” refers to the capability of precursor stem cells to differentiate into adipocytes, a process also known in the art as adipogenesis. Methods of inducing adipogenesis are known in the art and may contain steps of exposing said cell to transcription factors or chemical compounds that influence and modulate genetic, as well as protein, expression.

As used herein, the term “adipogenesis” refers to a process during which fibroblast like pre-adipocytes developed into mature adipocytes. Adipogenesis is understood in the art to be a well-orchestrated multistep process requiring the sequential activation of numerous transcription factors, including the CCAAT/enhancer-binding protein (C/EBP) gene family and peroxisome proliferator activated receptor-γ (PPAR-γ). In order to reach maturity, these cells must go through two vital steps: adipocyte determination and adipocyte differentiation. The stimulators involved in this process may include, but are not limited to, peroxisome proliferator-activated receptor γ (PPAR γ), insulin-like growth factor I (IGF-1), macrophage colony stimulating factor. (MCSF), fatty acids, prostaglandins and glucocorticoids Inhibitors may include glycoproteins, transforming growth factor-β (TGF-β), inflammatory cytokines and growth hormone.

The terms “hi”, “mid” and “lo”, as used herein, refer to the concentration of a certain indicator, with “lo” implying a reduced concentration of an indicator present and “hi” implying the presence of an increased concentration of indicator. That indicator could refer marker expression, as referred to herein, for example, the presence of a particular protein on the surface of a cell. Generally, the terms “hi”, “mid” or “lo” are used to indicate overall variability in cluster of differentiation (CD) expression, particularly when compared to unstained cells of the same cell type being studied. The concentrations implied therein, are measured in comparison to a benchmark defined by the experimenter, for example a ubiquitously expressed cell-surface protein. In one example, the percentage of cells expressing CD10 or other cell surface markers were determined by flow cytometry and compared to unstained controls. It was then shown that visceral adipose-derived stem cells express close to 0% of CD10⁺ cells.

As used herein the term “average” refers to the statistical or arithmetic mean, median or mode of a batch, sample, or distribution, or sometimes any other measure of central tendency, with the central tendency being understood as a statistically central value or a typical value for a probability distribution.

As used herein, the term “differentiation” refers to a process by which a functionally unspecialised cell becomes a more specialized cell. This process concerns cell types with the capability for differentiation known in the art, for example, but not limited to mesenchymal stem cells, adult and embryonic stem cells, organ-specific derived stem cells and progenitor cells. The differentiation ability, also known as “potency” in the art, of a cell then decreases in proportion to the stages of differentiation that have been completed by the same cell. Stages of potency may range from totipotent to pluripotent to multipotent to oligopotent to unipotent, with the potency decreasing with each stage. The differentiation of a cell from one stage to another may or may not be reversible and depend on cell type, conditions and technical manipulation known in the art.

Further disclosed herein is a method, as defined herein, wherein the method further comprises a step of isolating the adipose-derived stem cells. In one example, the stem cells referred to herein are isolated from an Asian sub-population.

The present invention also indicates towards the association of the identified markers with the adipogenic capability of cells isolation from subjects of differing ethnic descents or ethnic groups. The term “ethnic” as used herein relates to large groups of people classed according to common racial, national, tribal, religious, linguistic, or cultural origin or background. The term “race” is a term that was once commonly used in physical anthropology to denote a division of humankind possessing traits that are transmissible by descent and sufficient to characterize it as a distinct human type. Ethnic factors are used by the International Conference on Harmonization in a document (ICH ES: Ethnic Factors in the Acceptability of Foreign Clinical Data) that makes recommendations for strategies to permit clinical data collected in one region to be used to support drug and biologic registrations in another region while allowing for the influence of ethnic factors. In this document, ethnic populations are classified as Asian, Black and Caucasian. For example, in its draft guideline recommendations, the Federal Drug Administration (FDA) requires an analysis of data according, inter alia, to demographic subgroups (age, gender, race). For ethnicity, the FDA recommends Hispanic or Latino and not Hispanic or Latino as the minimum choice to be offered to trial participants. For race, the minimum choices are American Indian or Alaska Native, Asian, Black or African American, Native Hawaiian or Other Pacific Islander and White.

As used herein, the term “isolating”, which may also be replaced with the term “harvesting” or “recovering”, refers to the process of extracting the desired cell, cell type or components of a tissue from its surrounding matrix that is commonly associated with the cell or components of a tissue. For example, the term “isolating” in this instance refers to the act of extracting the adipose-derived stem cell from the method of the present disclosure from, for example, adipose tissue.

In one example, the method as defined herein, is disclosed, wherein the stem cells are isolated using a method selected from a group consisting of enzymatic digestion, mechanical dissociation, chemical dissociation, antibody binding, agitation, cell-sorting, fluorescent-activated cell sorting (FACS) and combinations thereof. In another example, there is disclosed a method of using cell surface markers, as defined herein, wherein the cell surface markers are used in a high-throughput screening assay for drugs or compounds that would enhance adipogenesis. In a further example, the high-throughput screening assay would assay for compounds that convert visceral adipose-derived stem cells into a subcutaneous adipose-derived stem cell-like phenotype.

Known methods in the art for isolating targeted cell types may involve dissociation enzymes such as collagenase, for enzymatic digestion. Other methods in the art include mechanical dissociation, which is the use of physical force for dissociating the surrounding tissue, chemical dissociation, antibody binding, agitation, cell-sorting, fluorescent-activated cell sorting (FACS) and combinations thereof. The term “antibody binding” refers to methods that involve antibody-target cell interaction for isolation of the same. These methods listed here can be understood as positive or negative selection, which is positive selection being the isolation of target cells out of a cell mix and negative selection being the removal of the cell mix and the retention of target cells.

As used herein the term “enzymes” refers to biological molecules that are capable of digesting extracellular matrix that binds cells together to form tissue. In the present disclosure, the enzymes may be dissociating enzymes that facilitate the digestion or dissociation of cell-to-cell contact such as collagenase, trypsin, and the like that can facilitate digestion of a tissue to single cell suspension. As used herein, the term “digestion”, “digesting”, “dissociation” or any other grammatical permutations that can be used interchangeably in the art, refers to the process of breaking down extracellular matrix that binds cells together to form tissue. Other enzymes that may be used in the context may be chymosin, collagenase type 1, collagenase type 2, collagenase type 3, collagenase type 4, deoxyribonuclease I (DNasee, deoxyribonuclease II (DNasell), dispase, elastase, hyraluronidase, ovomucoid protease inhibitor, pancreatin, papain, pronase, protease, trypsin, soybean trypsin inhibitor and combinations thereof.

As used herein, the term “high-throughput screening assay” refers to a drug-discovery process widely used in the pharmaceutical industry. It utilizes assay and liquid handling automation to quickly test biological or biochemical activity of a large number of drugs or drug-like compounds. These compounds and drugs may tested for targets, including ligands for receptors, enzymes, ion-channels or other pharmacological targets, or pharmacologically profiling a cellular or biochemical pathway of interest. In one example, the compounds, as defined herein, may enhance adipogenesis. In another example, the compounds, as defined herein, may convert cells with dysfunctional adipogenic capacity into cells with functional adipogenic capacity.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Example 1: Isolation and Culture of Adipose-Derived Stem Cells

The herein disclosed cell surface marker analyses were performed on subcutaneous adipose-derived stem cells, which are easily and routinely obtained by liposuction procedure.

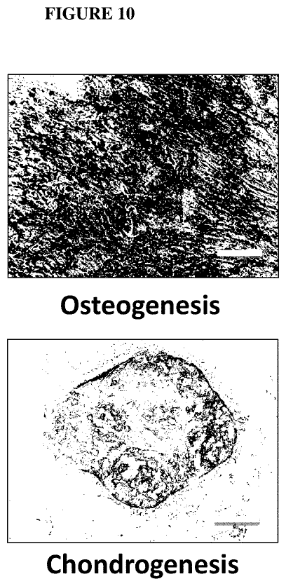

In this case, white adipose tissue was isolated from the subcutaneous (abdominal region) and visceral (omental region) depots from a cohort of seven human obese volunteers (subjects S1 to 12), with body mass index (BMI) of more than 30, undergoing bariatric surgery (Table 1). This was done with informed consent from all subjects and approved by the NHG Domain Specific Review Board (DSRB) at National Healthcare Group, Singapore. Adipose-derived stem cells were then isolated from the white adipose tissue by collagenase digestion and cultured, up to passage 10, in high glucose Dulbecco's modified Eagle's medium (DMEM) supplemented with 15% fetal bovine serum, non-essential amino acid, penicillin streptomycin and 5 ng/ml basic human fibroblast growth factor (bFGF) as described previously (Sugii et al., 2011). All cells were maintained at 37° C. in a 5% CO₂ humidified incubator. The medium was changed every 2-3 days. Adipose-derived stem cells were checked for their multipotent capacity by osteogenesis (STEMPRO Osteogenesis Differentiation Kit) and chondrogenesis (STEMPRO Chondrogenesis Differentiation Kit) assays, followed by Alizarin Red S and Alcian Blue stainings, respectively (FIG. 10), in addition to adipogenesis described below.

TABLE 1
Donor information

| Subject | Age | Sex | Ethnic group |
|---|---|---|---|
| S1 | 43 | F | Malay |
| S2 | 36 | M | Malay |
| S3 | 54 | F | Indian |
| S4 | 51 | F | Malay |
| S5 | 53 | M | Malay |
| S6 | 35 | F | Malay |
| S7 | 51 | F | Malay |
| S8 | 39 | F | Chinese |
| S9 | 26 | F | Malay |
| S10 | 34 | F | Chinese |
| S11 | 37 | M | Chinese |
| S12 | 25 | F | Chinese |

### Example 2: High Content Screening

In order to study molecular marker differences of stem cell populations of different depots, stromal vascular fractions of subcutaneous (SC) and visceral (VS) fat depots were isolated and adipose-derived stem cells were enriched by serial passage culture of the stromal vascular function as previously described. The approach consisted of three stages in order to identify depot-specific adipose-derived stem cell markers (FIG. 1A). High content screening assay of 242 human cell surface markers using BD Lyoplate Human Cell Surface Marker Screening Panel (BD Biosciences) was performed on subcutaneous and visceral adipose-derived stem cells from subject S1, commercial subcutaneous adipose-derived stem cells (Lonza PT-5006), bone-marrow-derived-mesenchymal stem cells (Lonza PT-2501) and human foreskin fibroblasts HFF-1 (ATCC SCRC-1041) according to the manufacturer's manual.

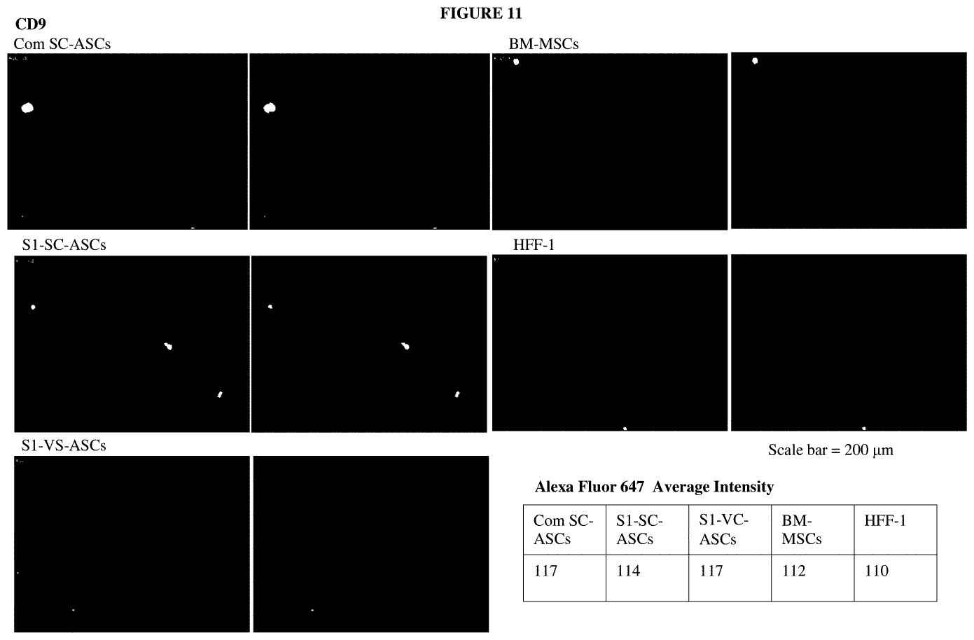

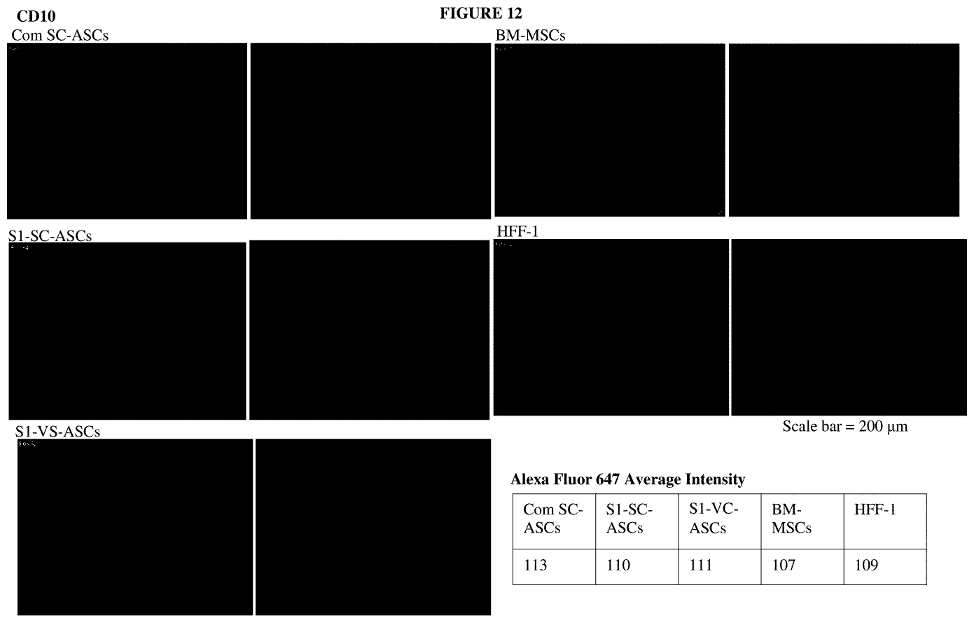

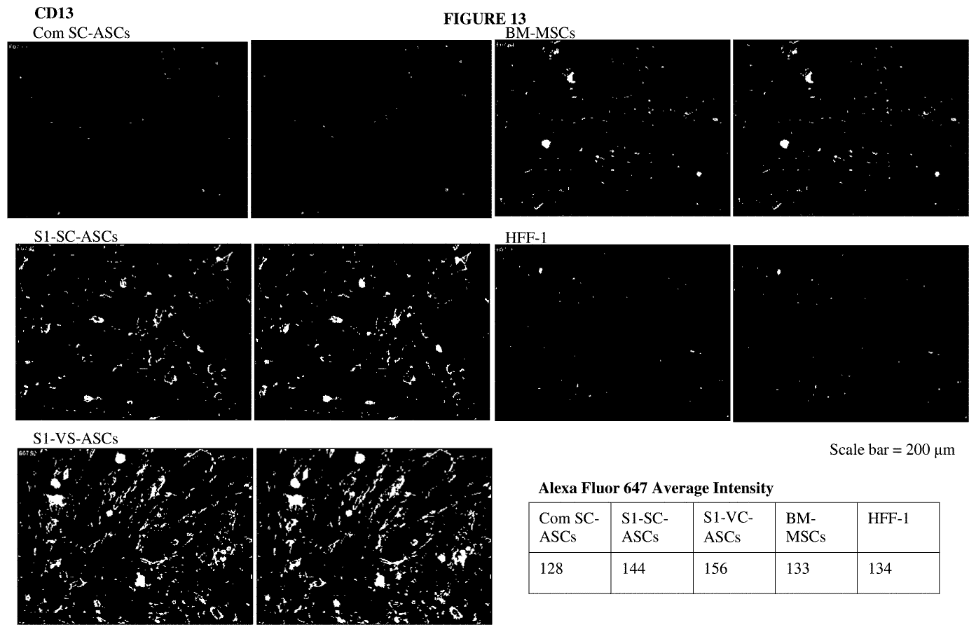

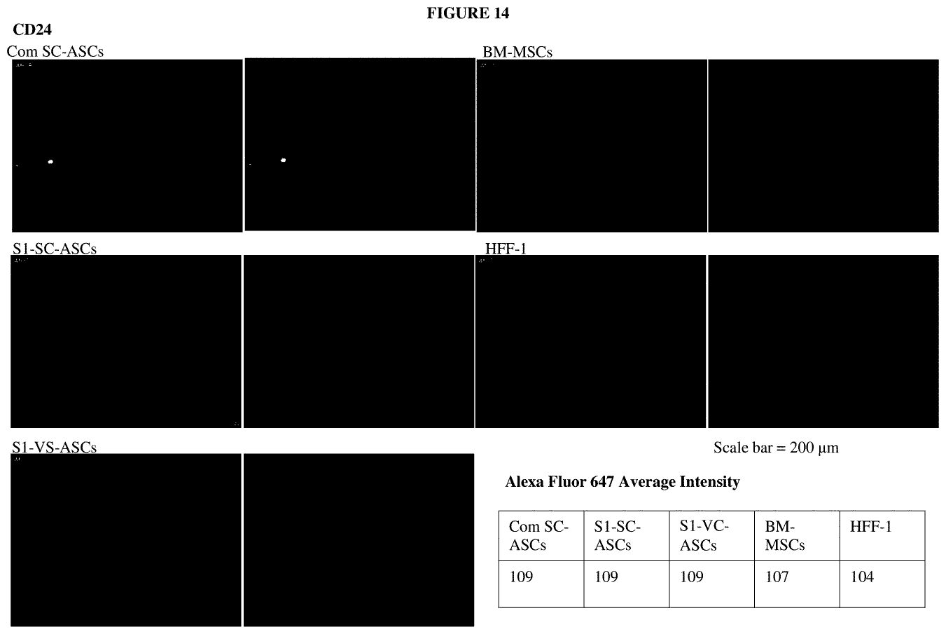

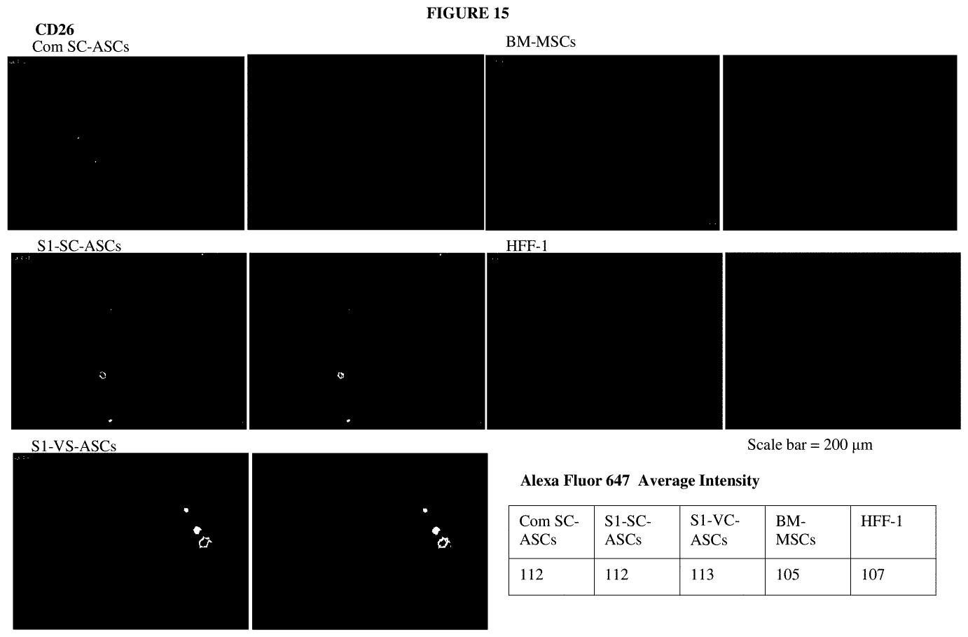

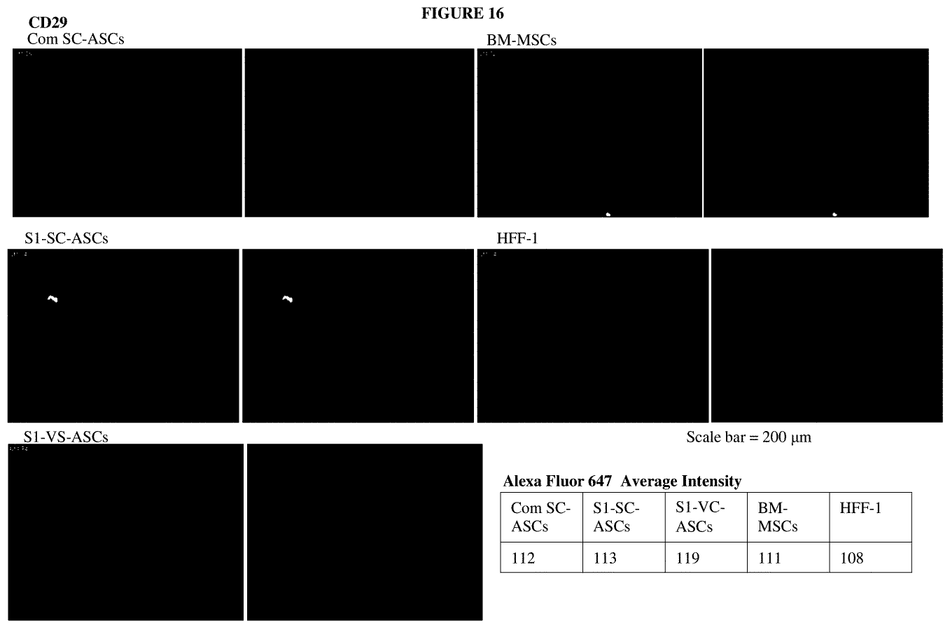

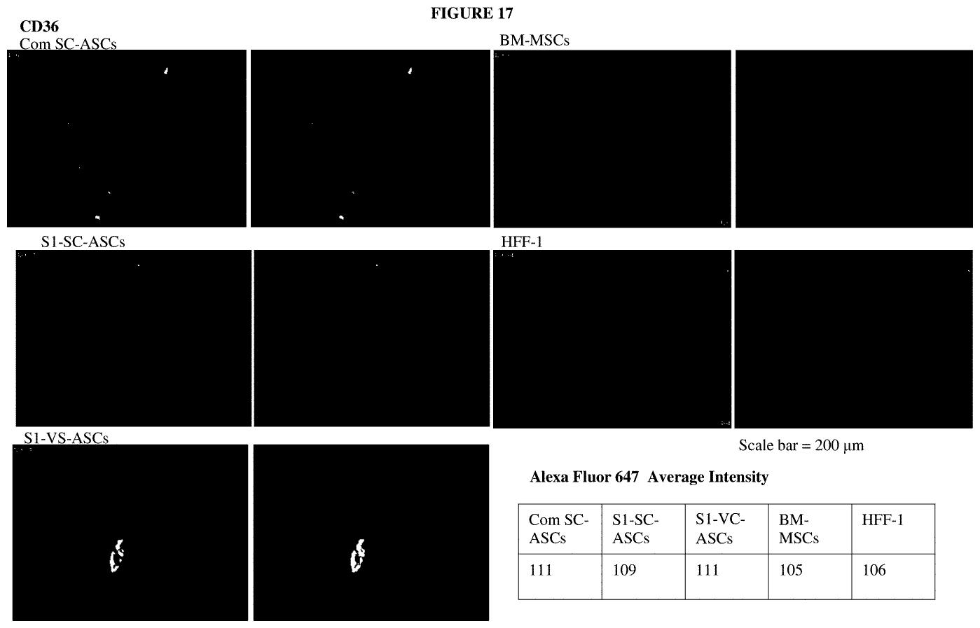

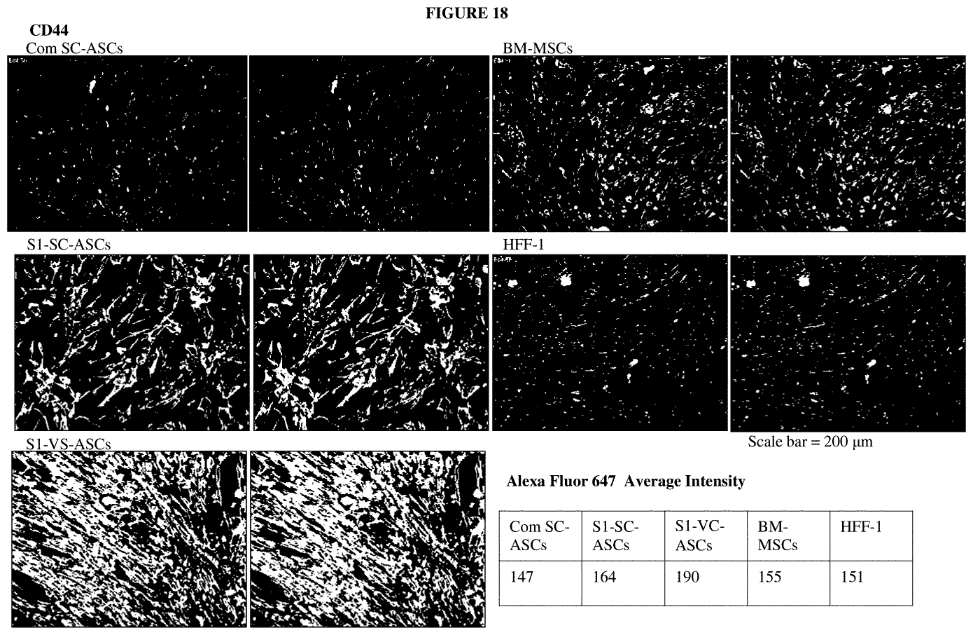

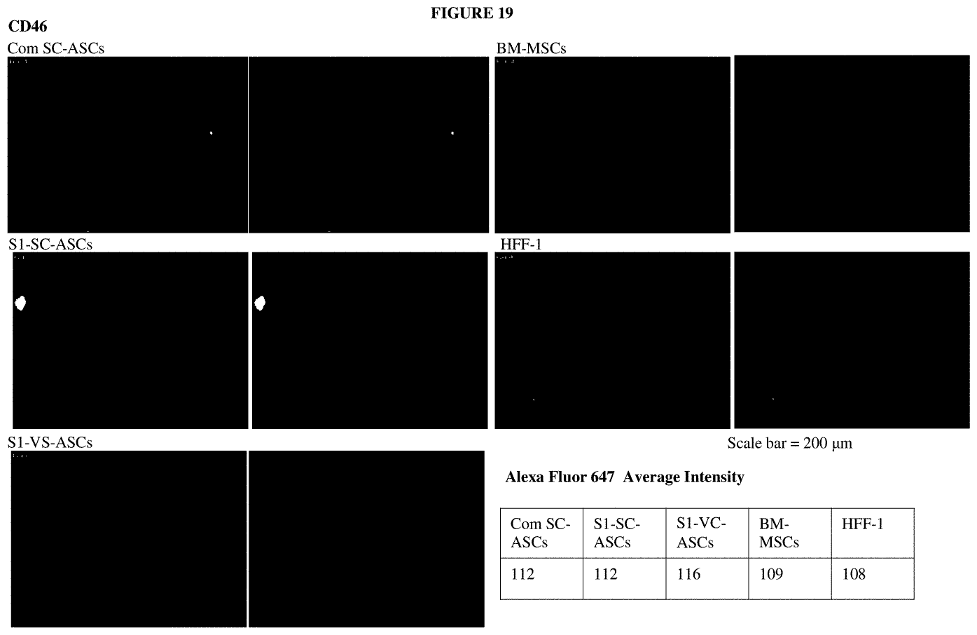

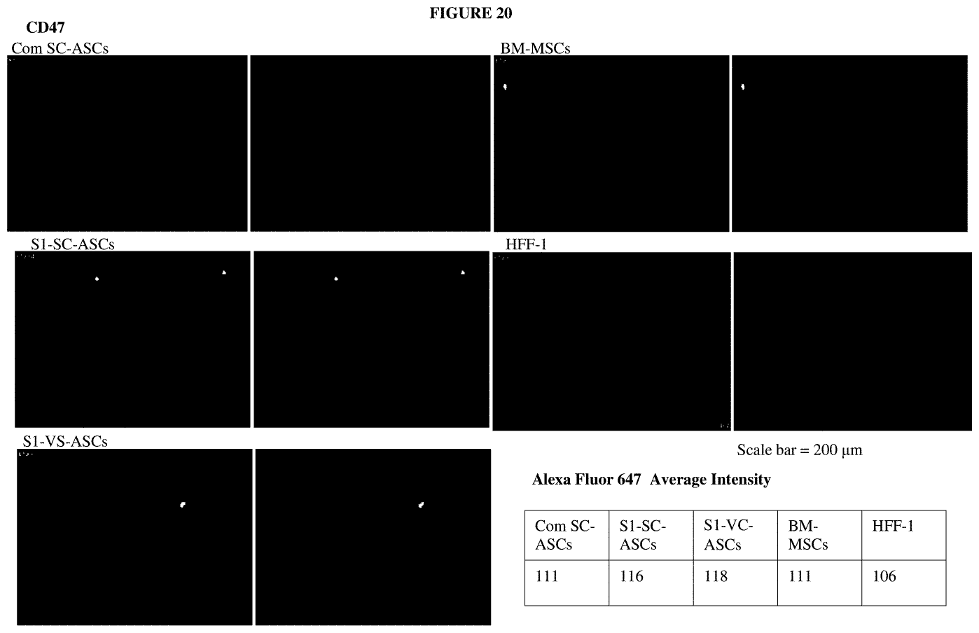

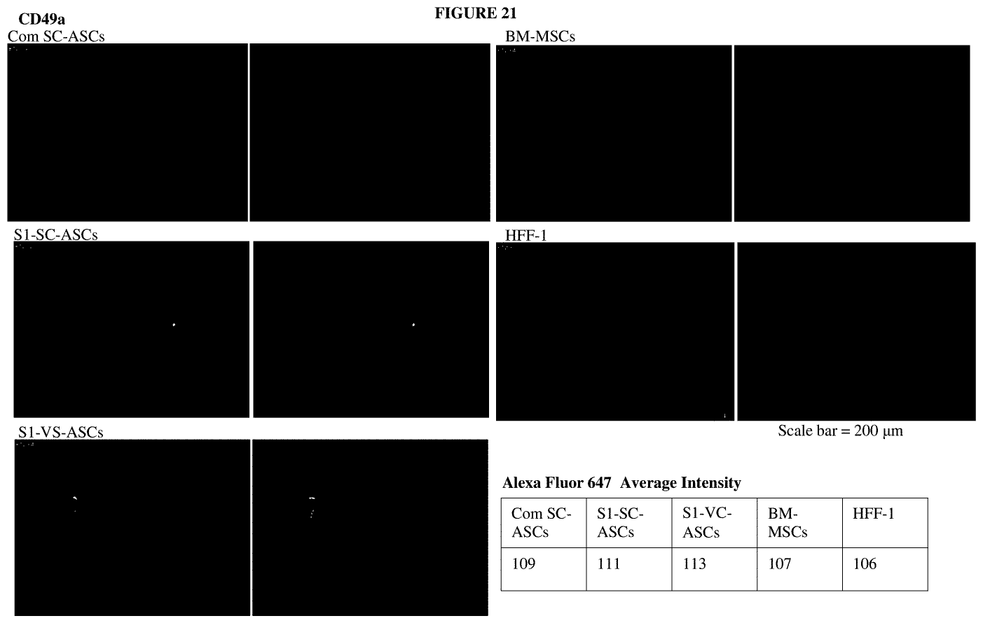

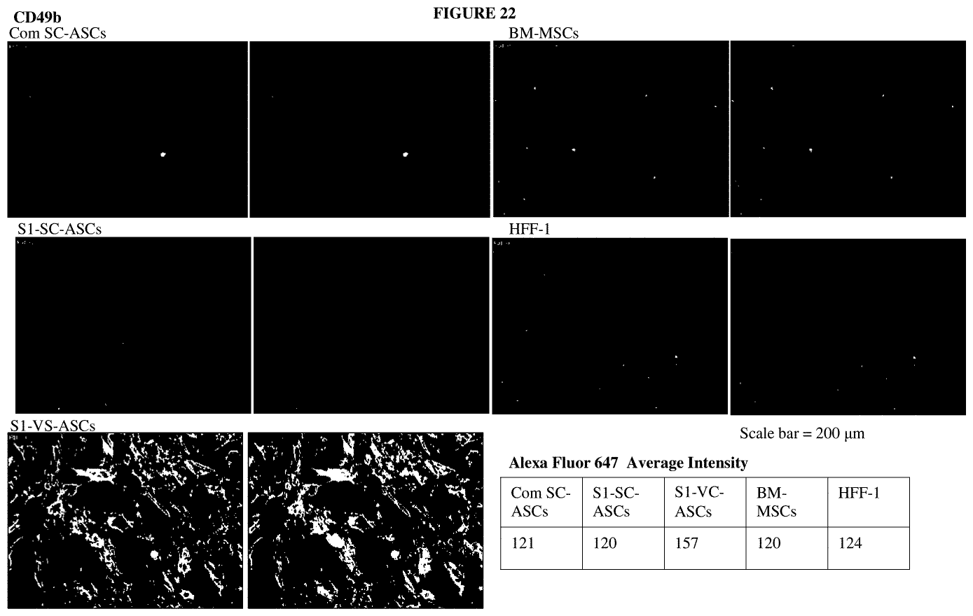

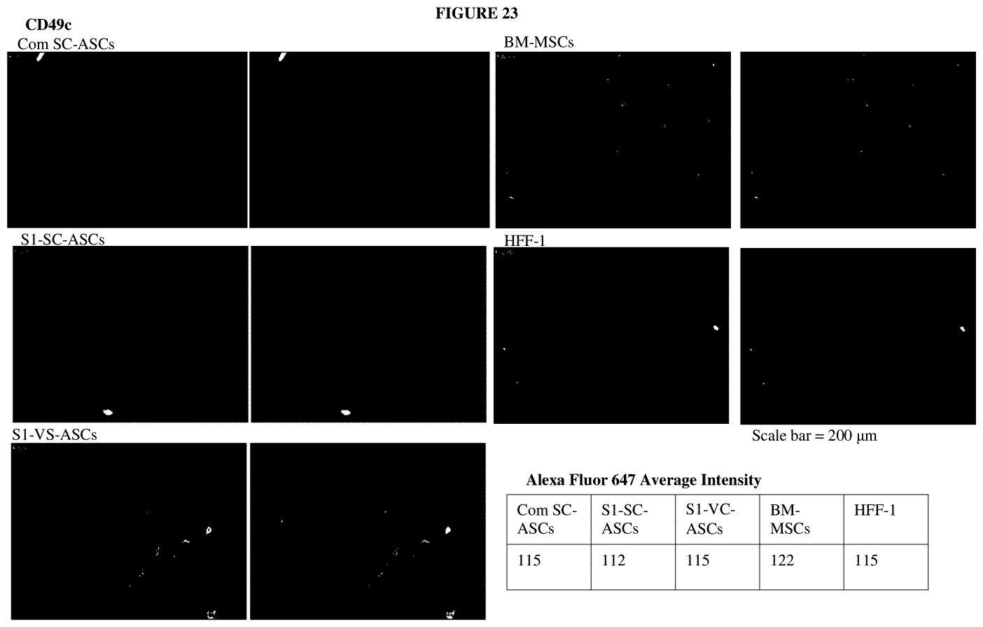

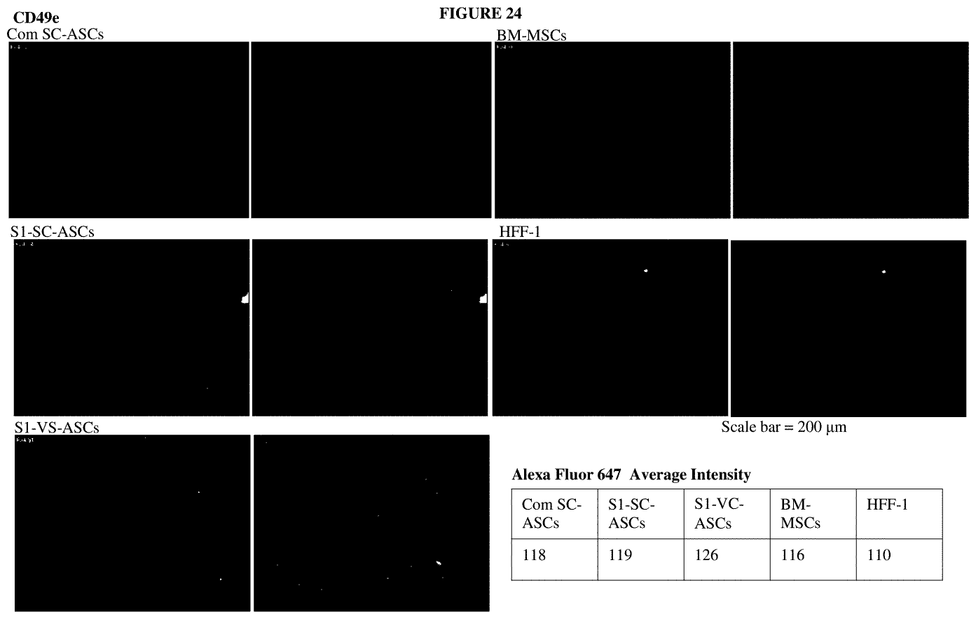

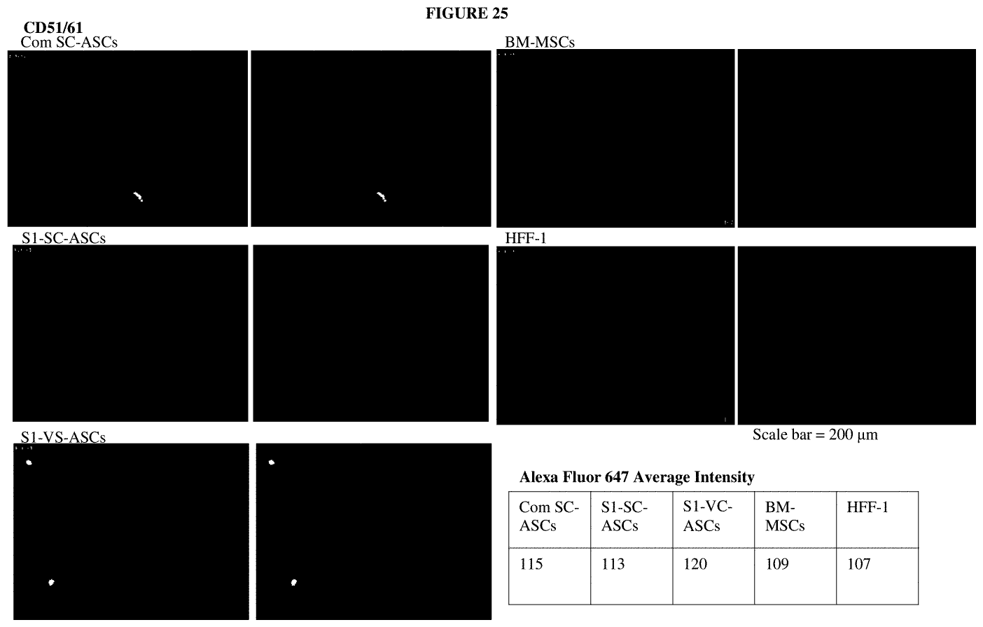

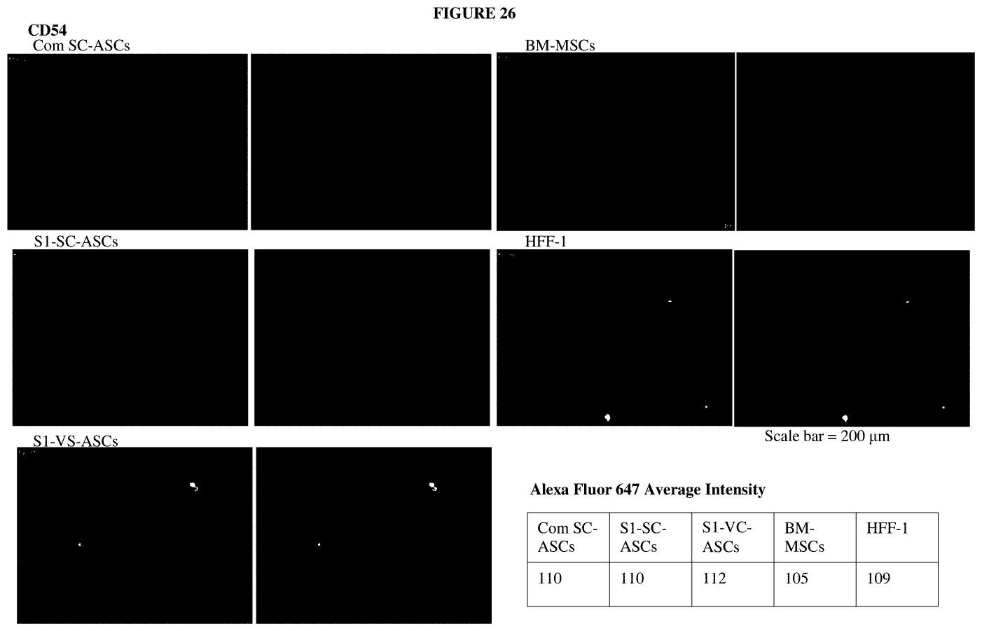

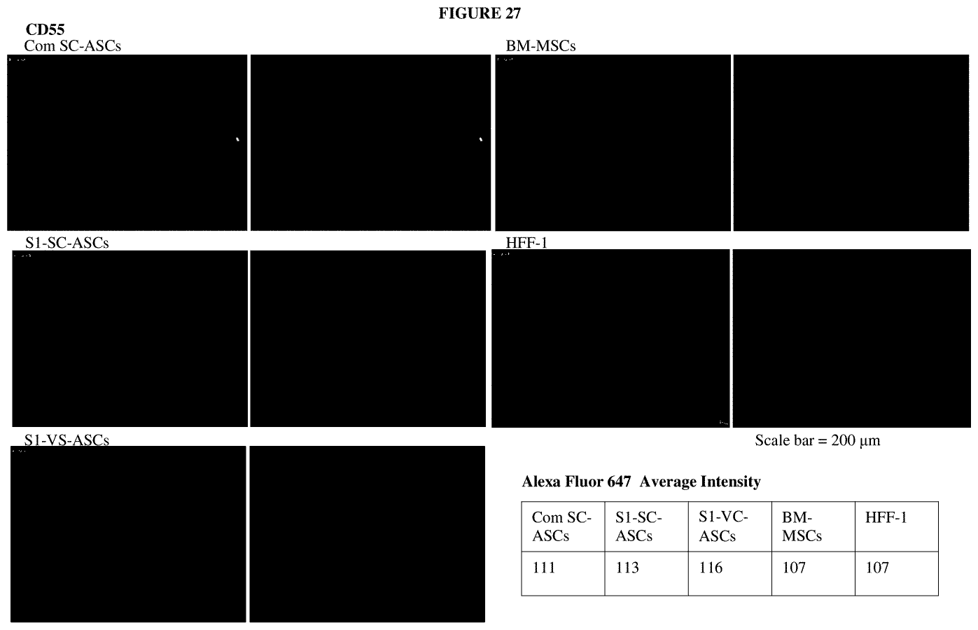

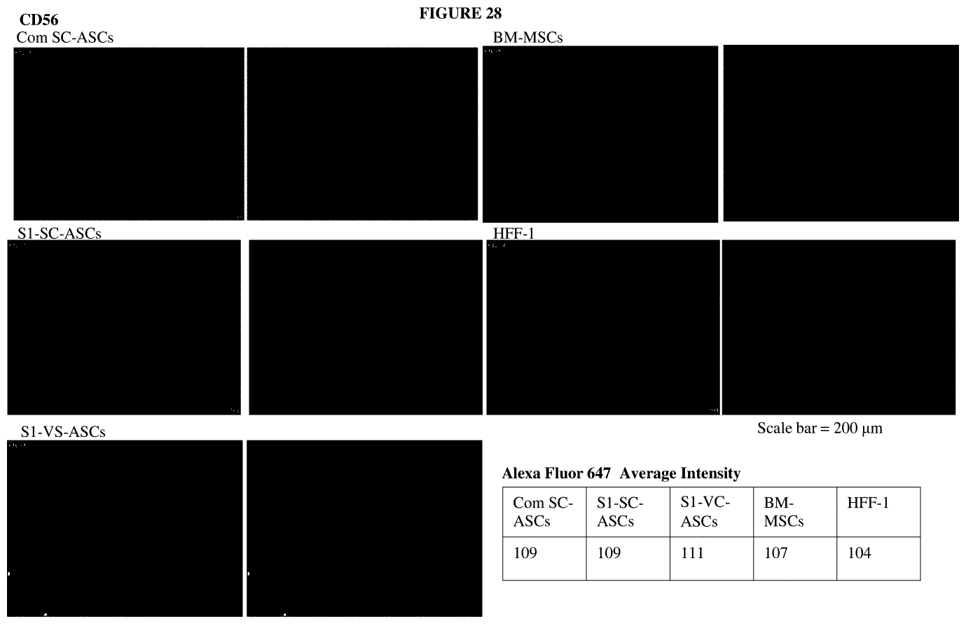

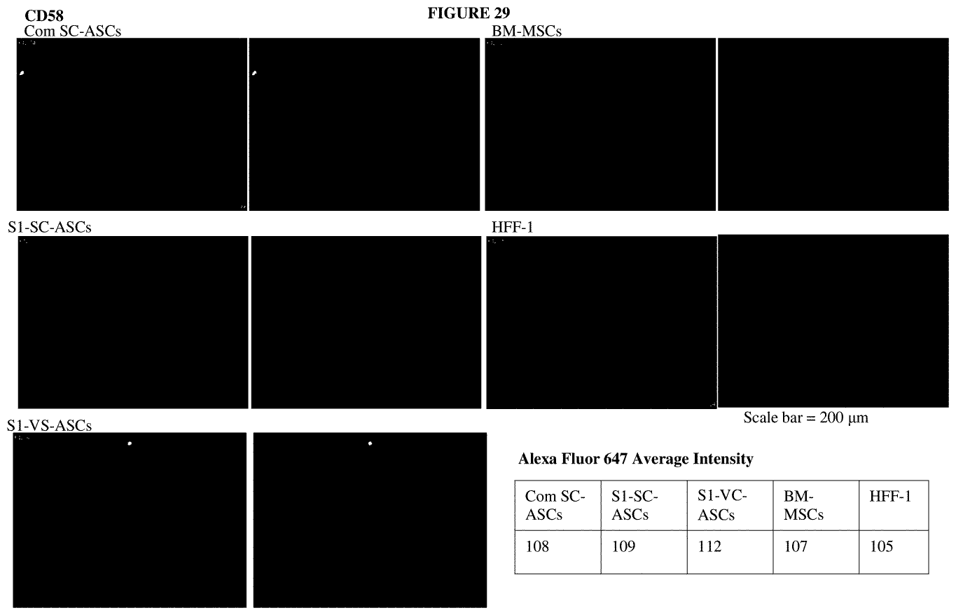

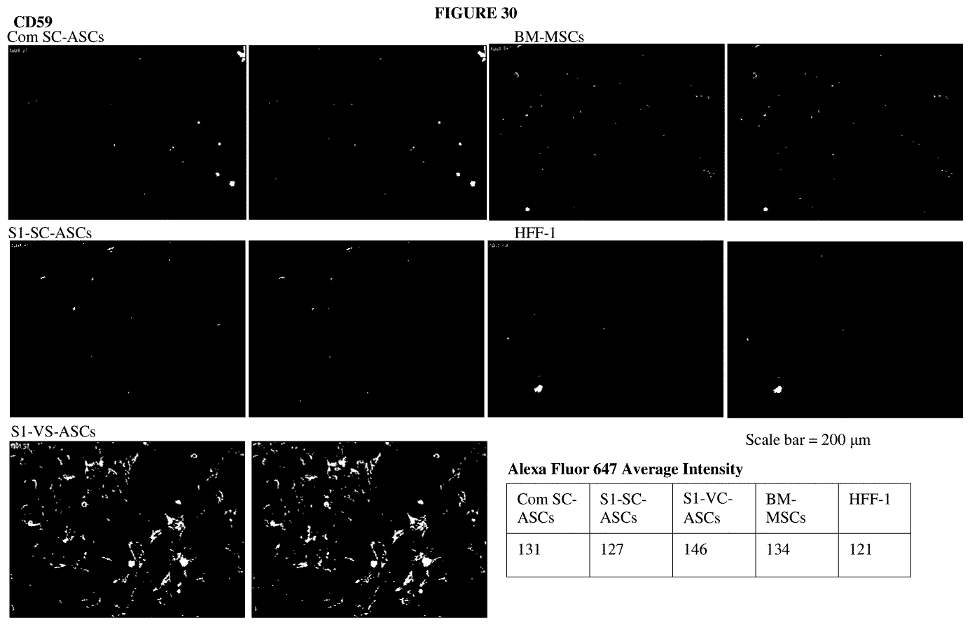

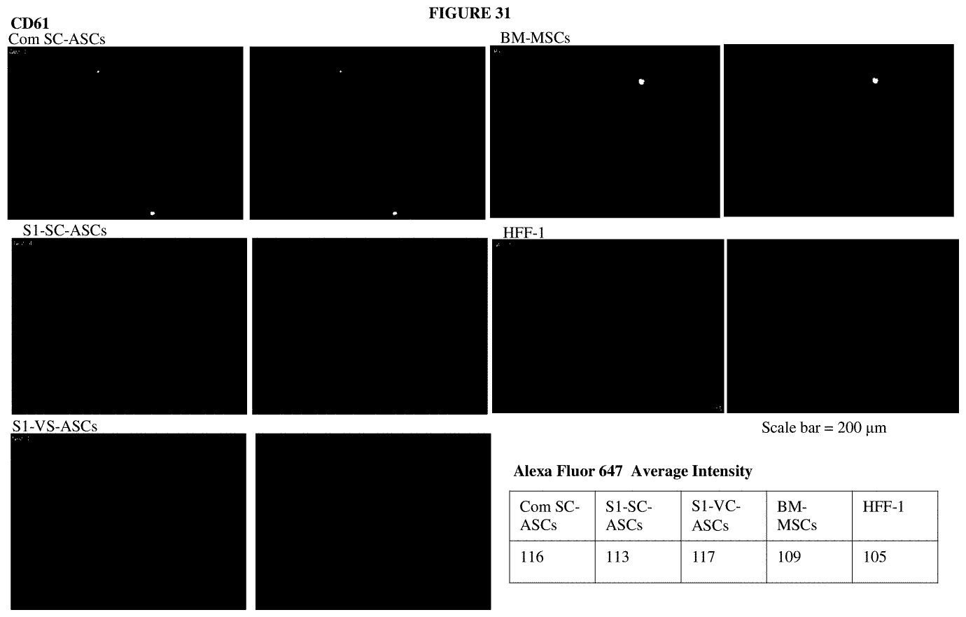

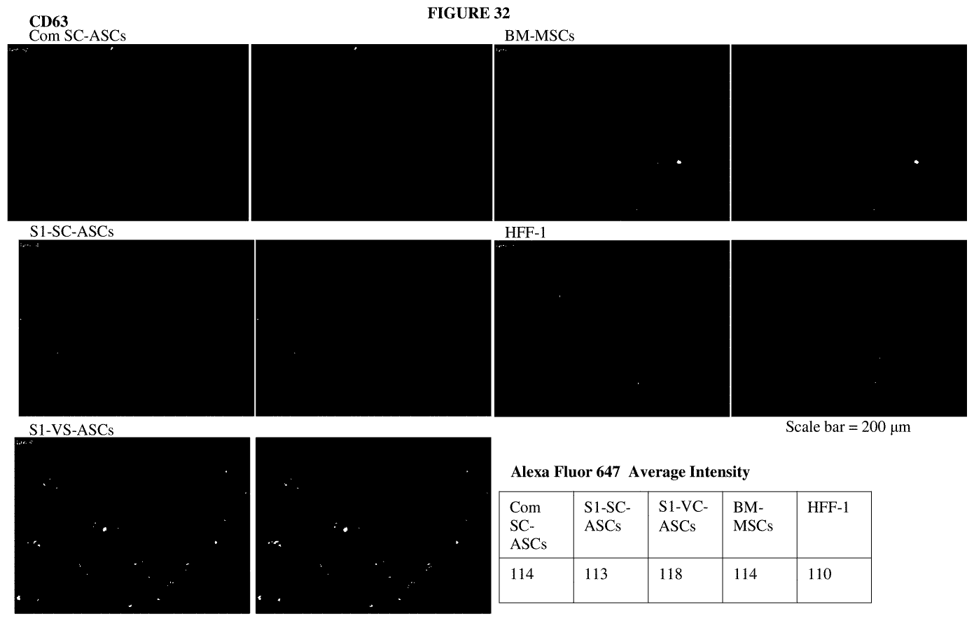

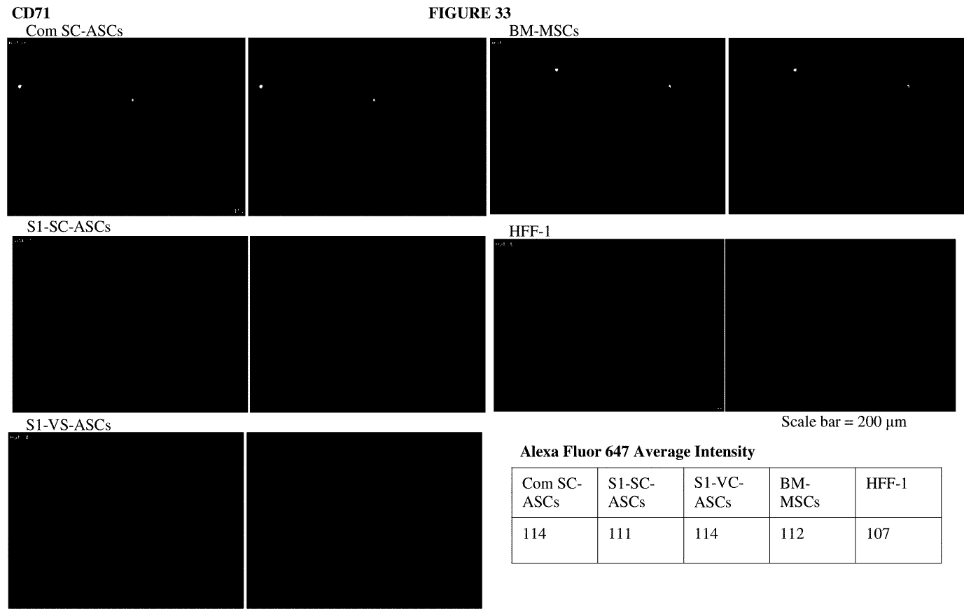

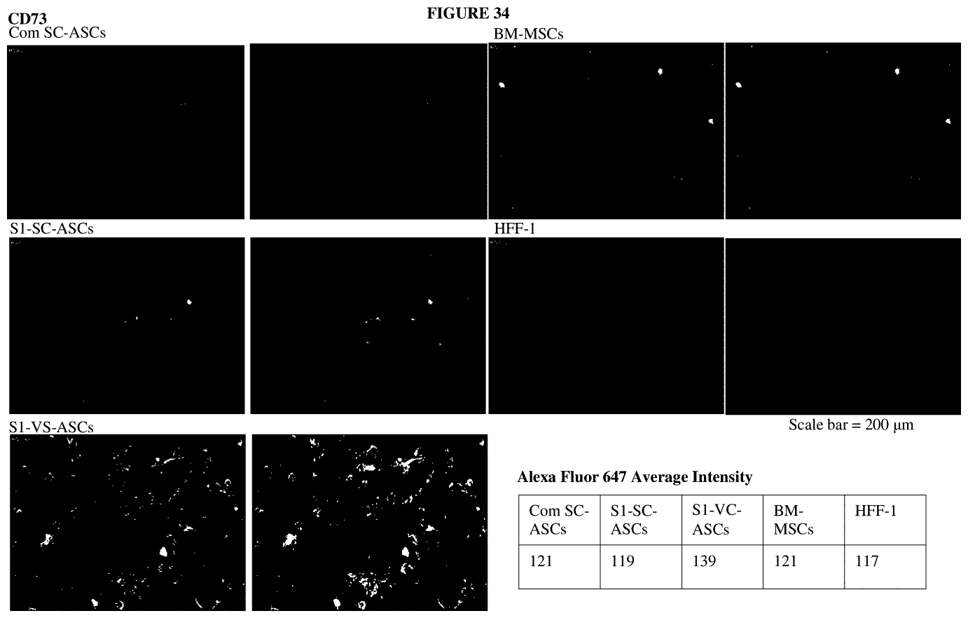

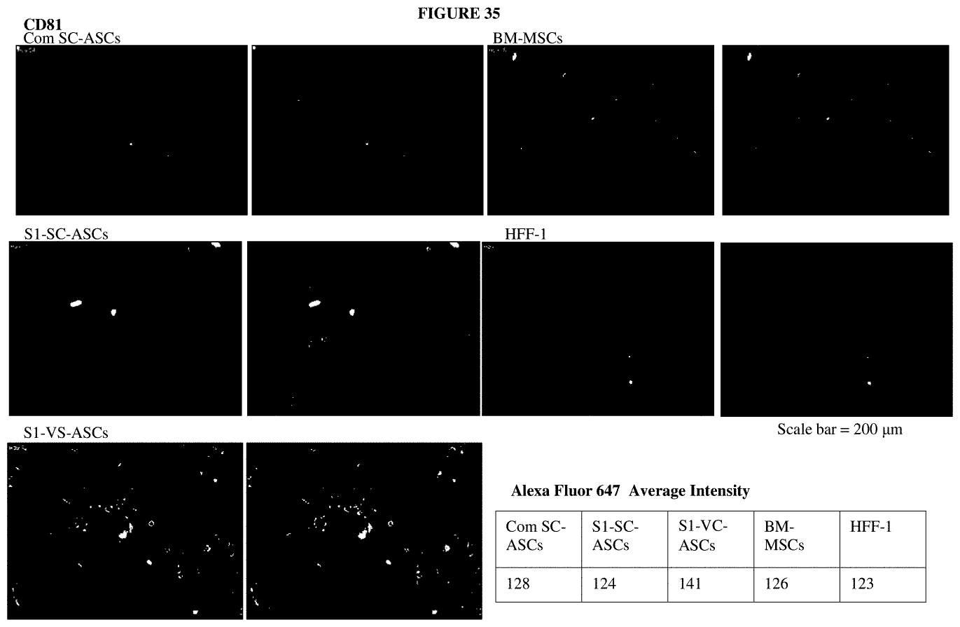

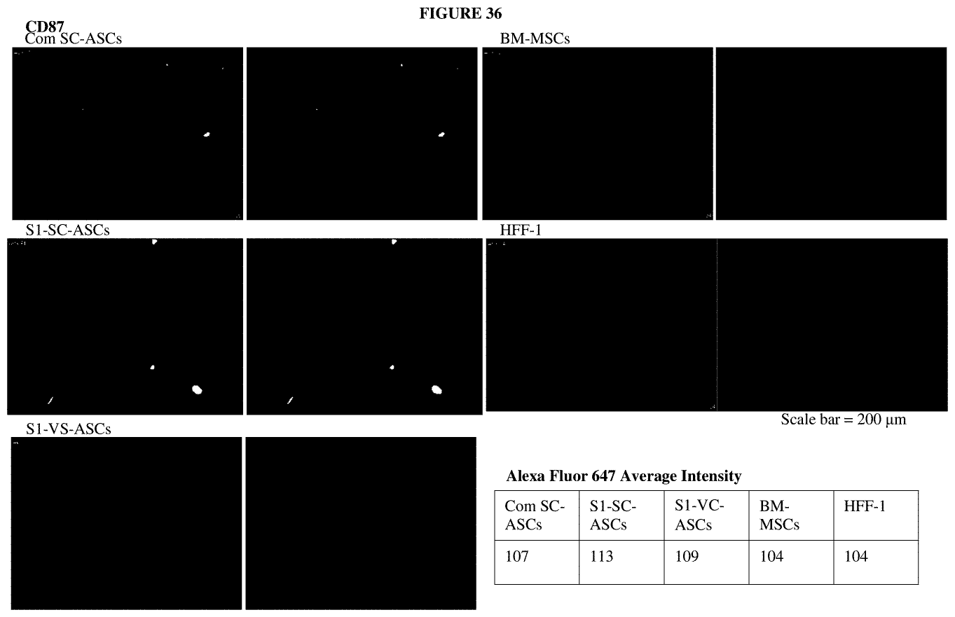

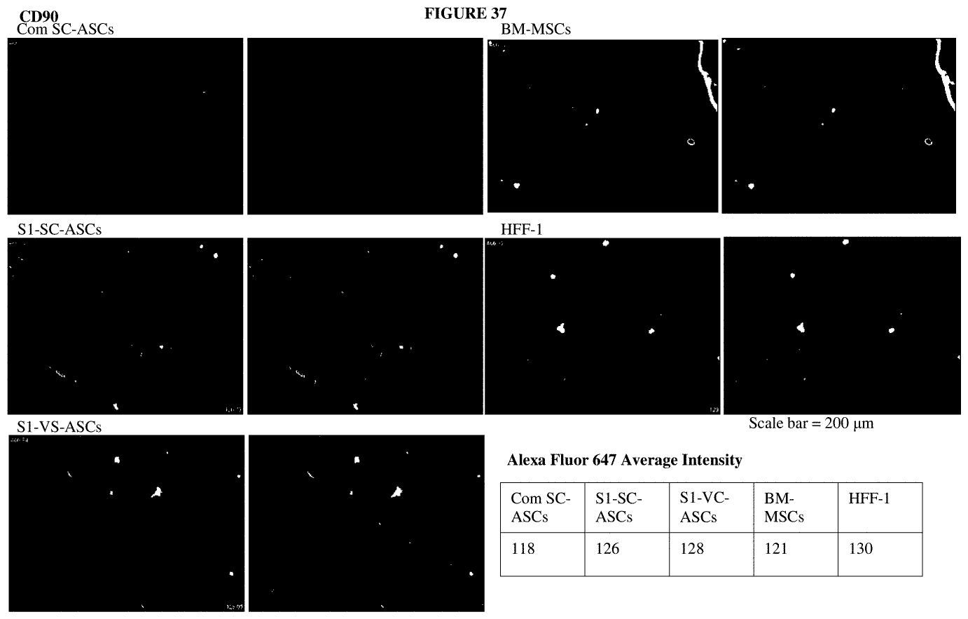

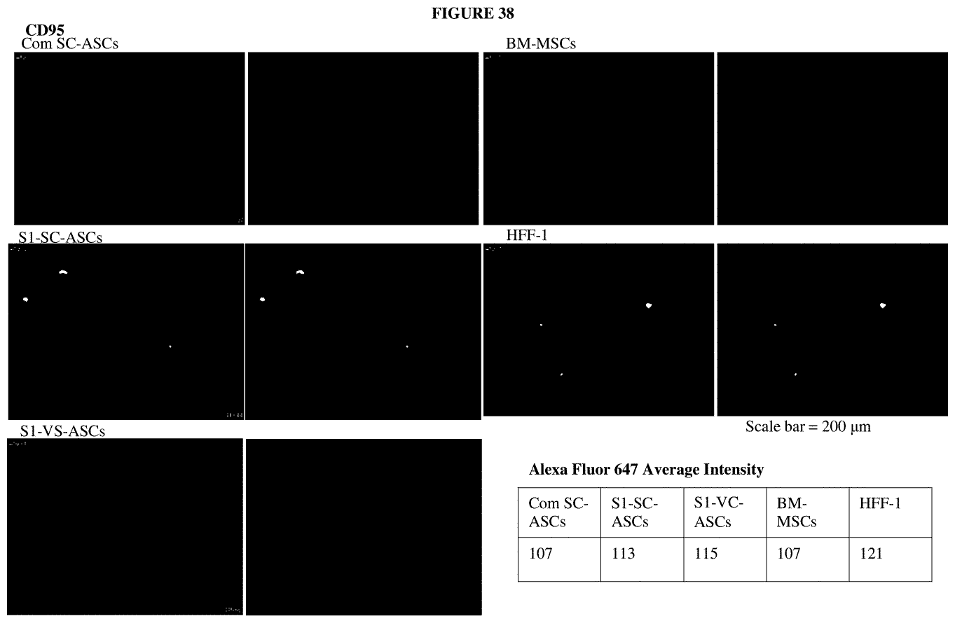

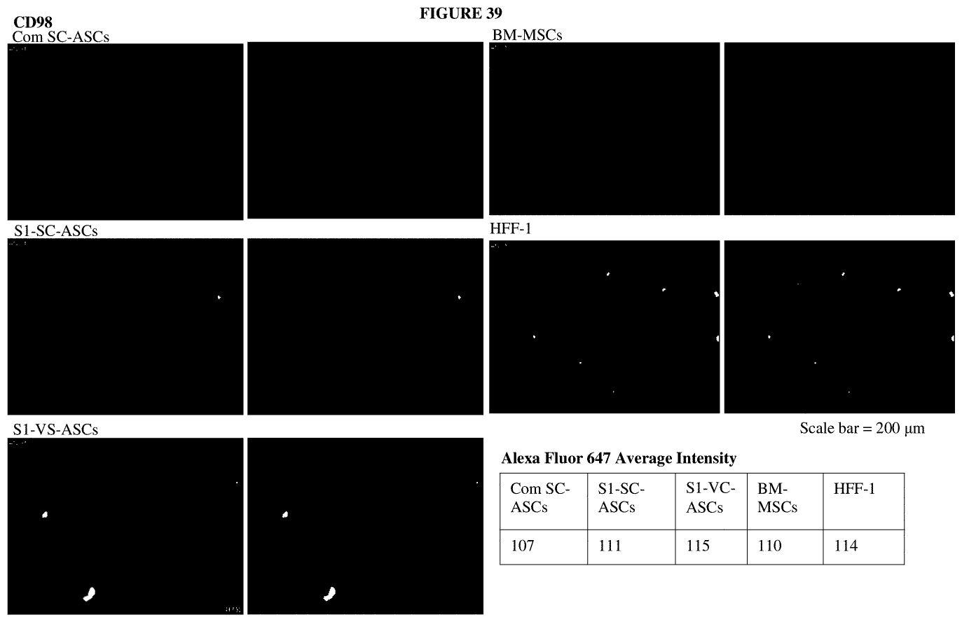

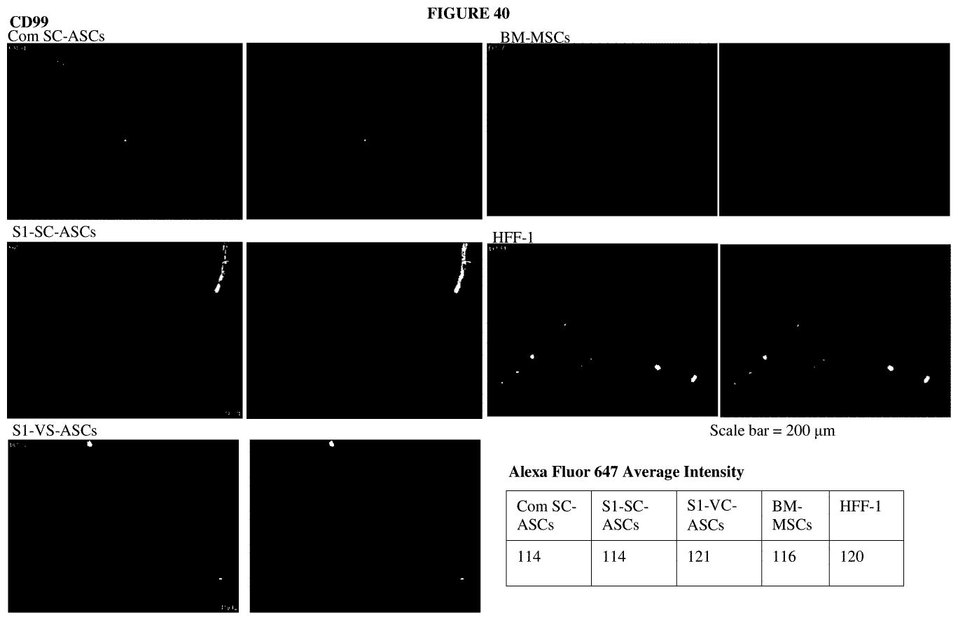

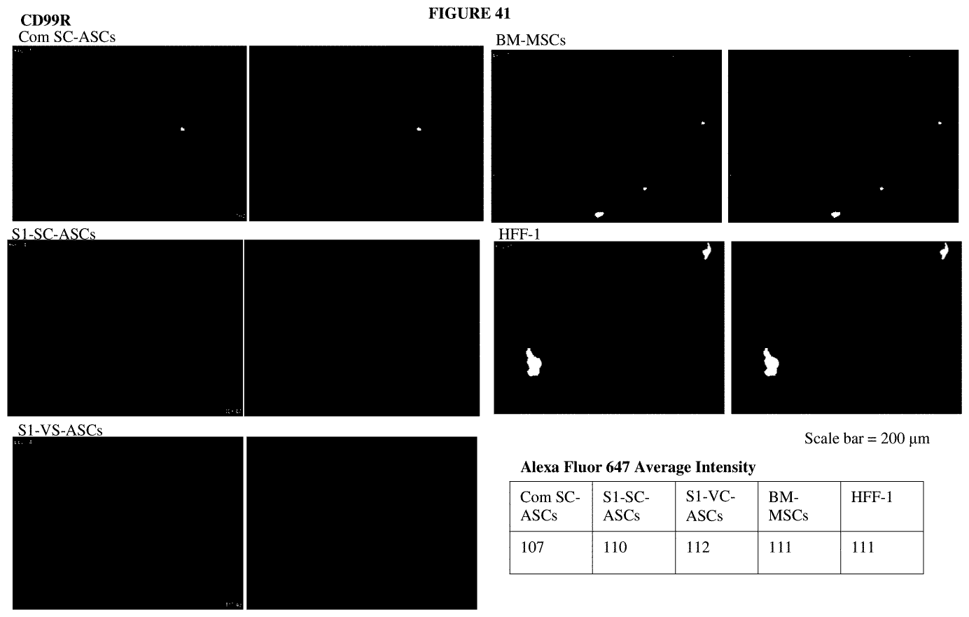

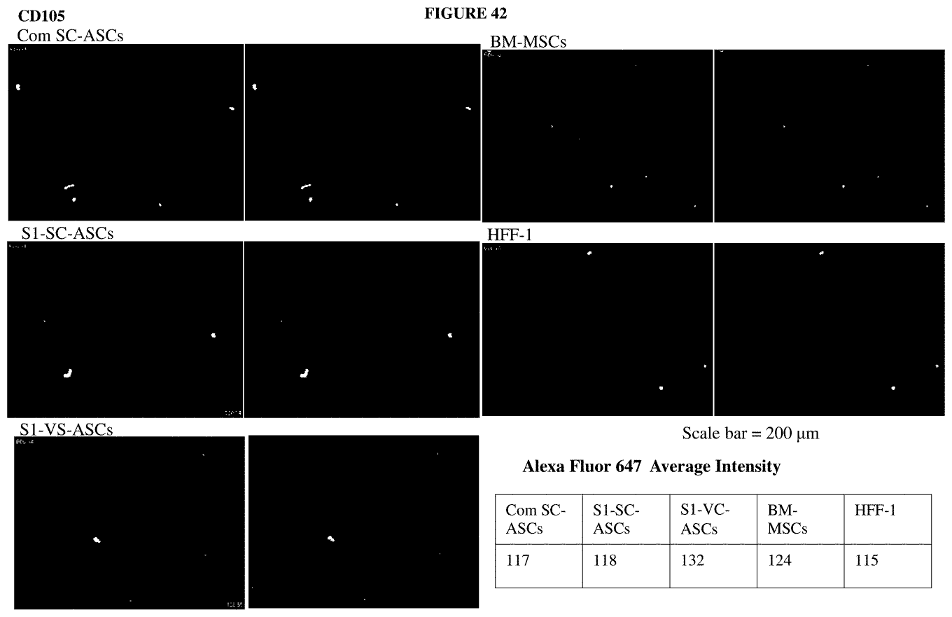

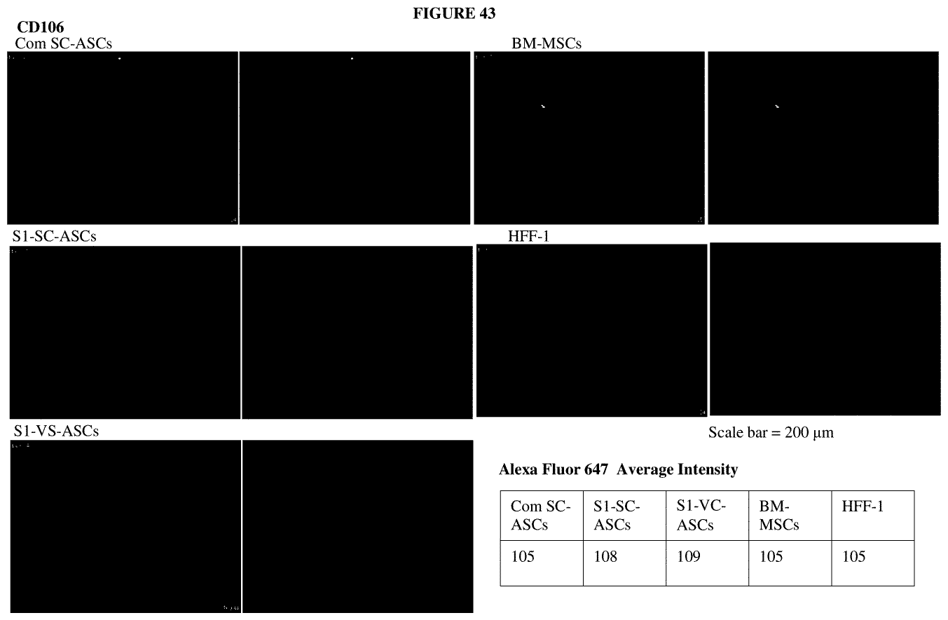

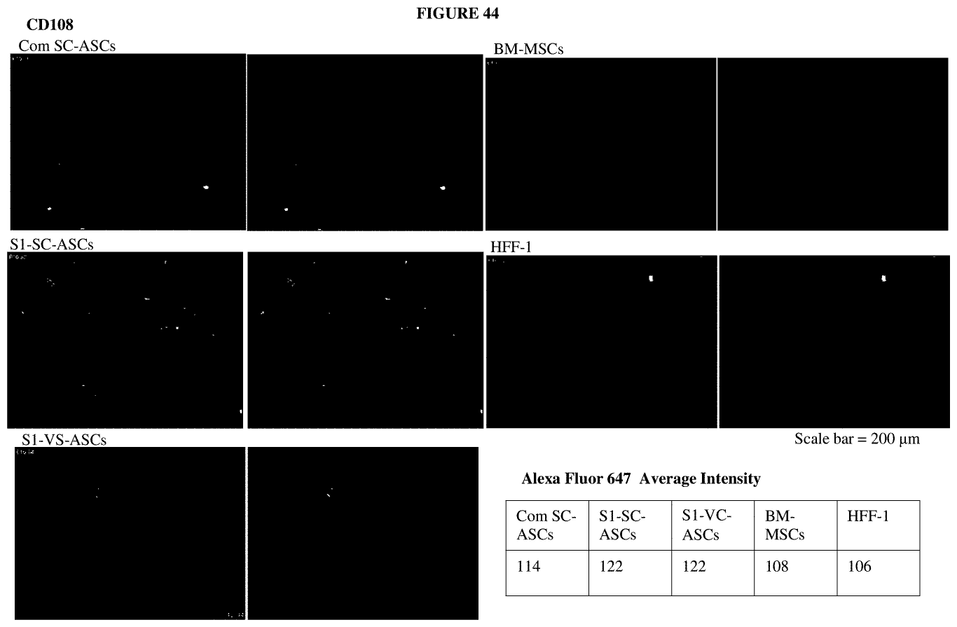

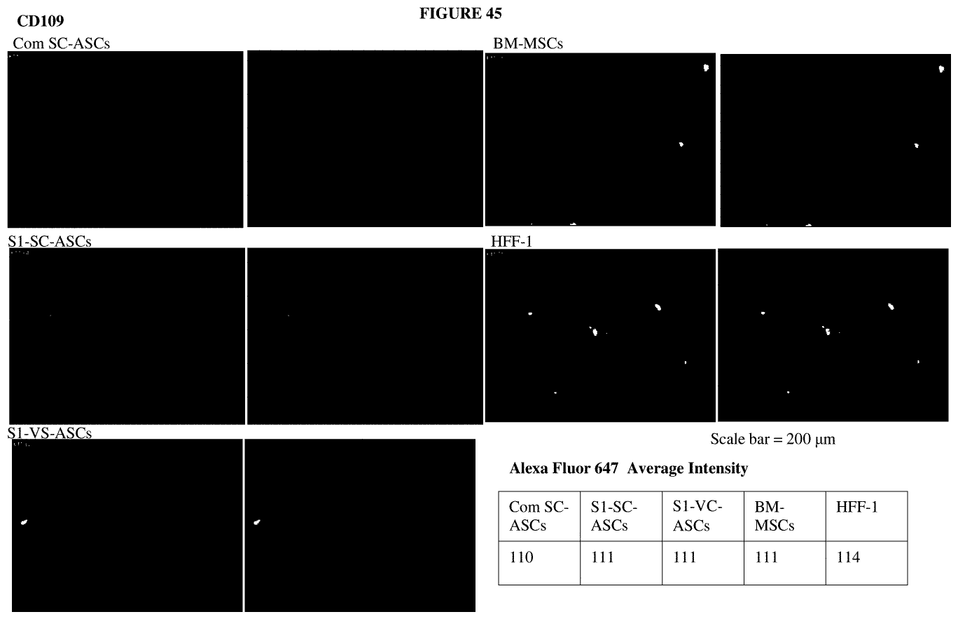

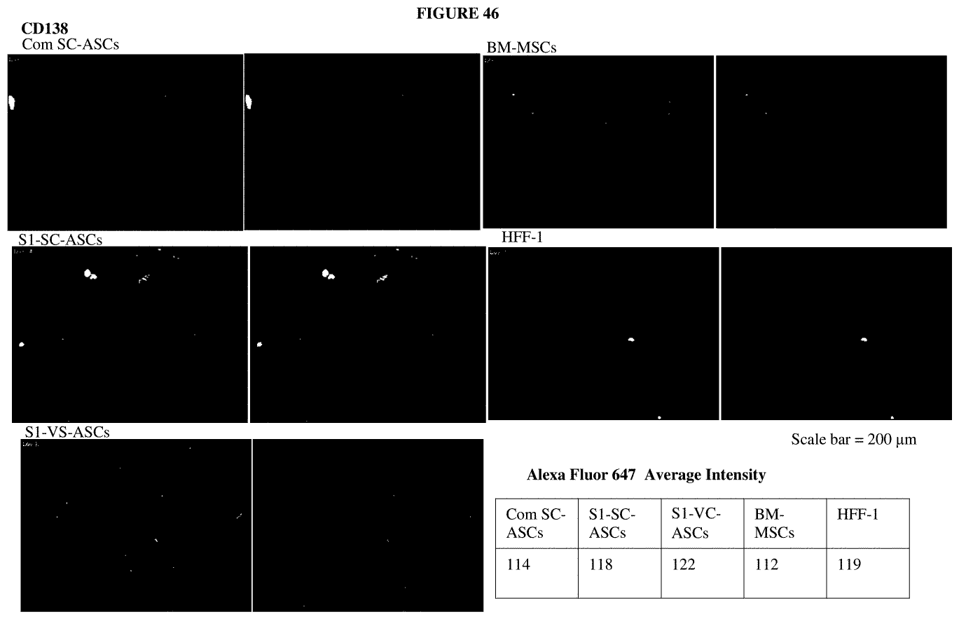

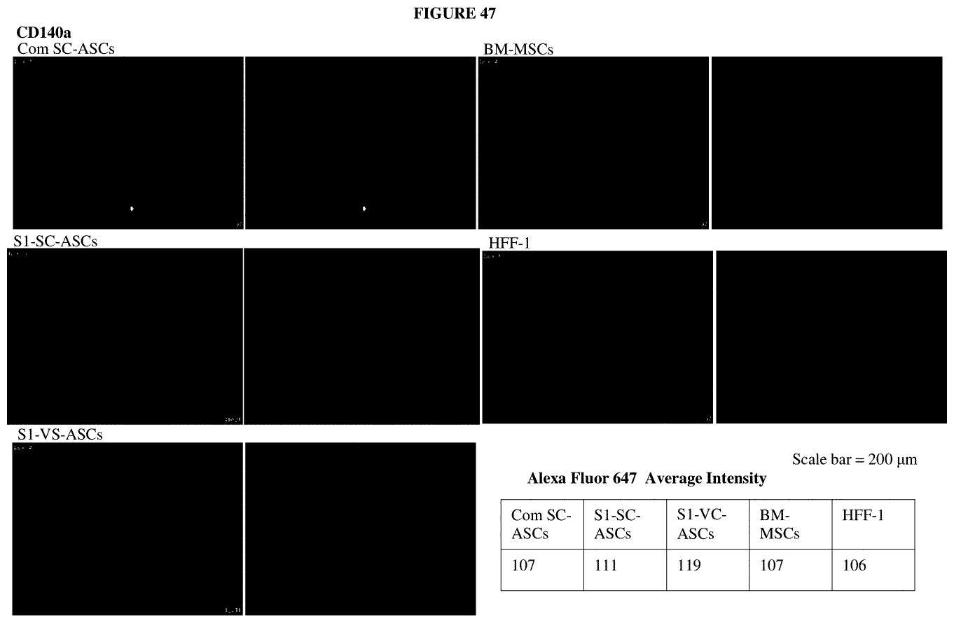

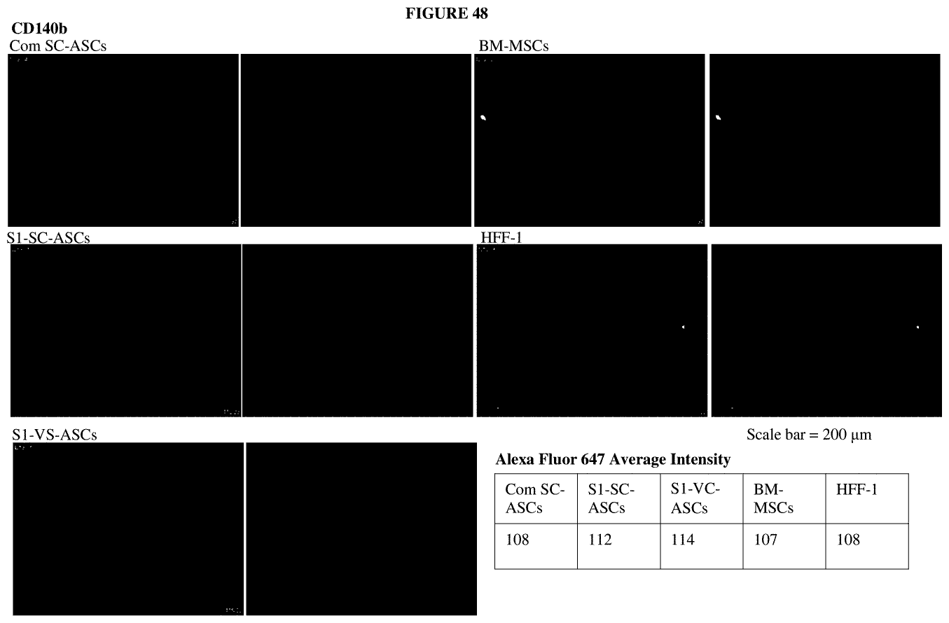

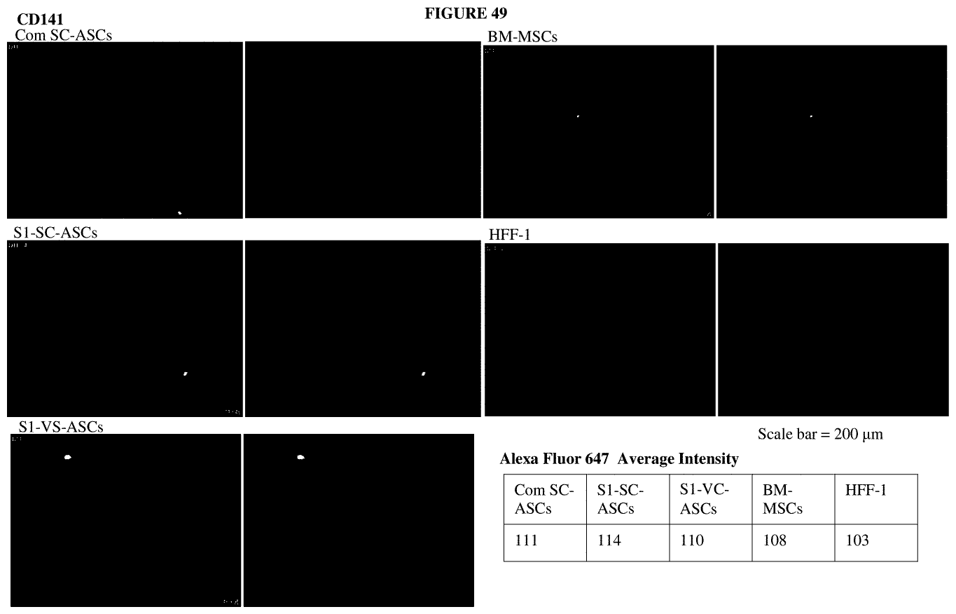

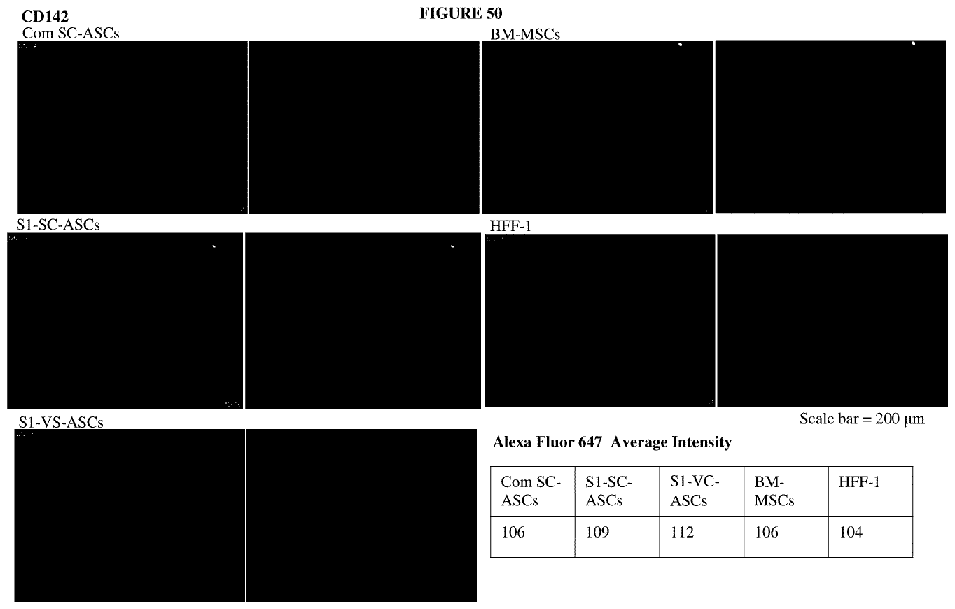

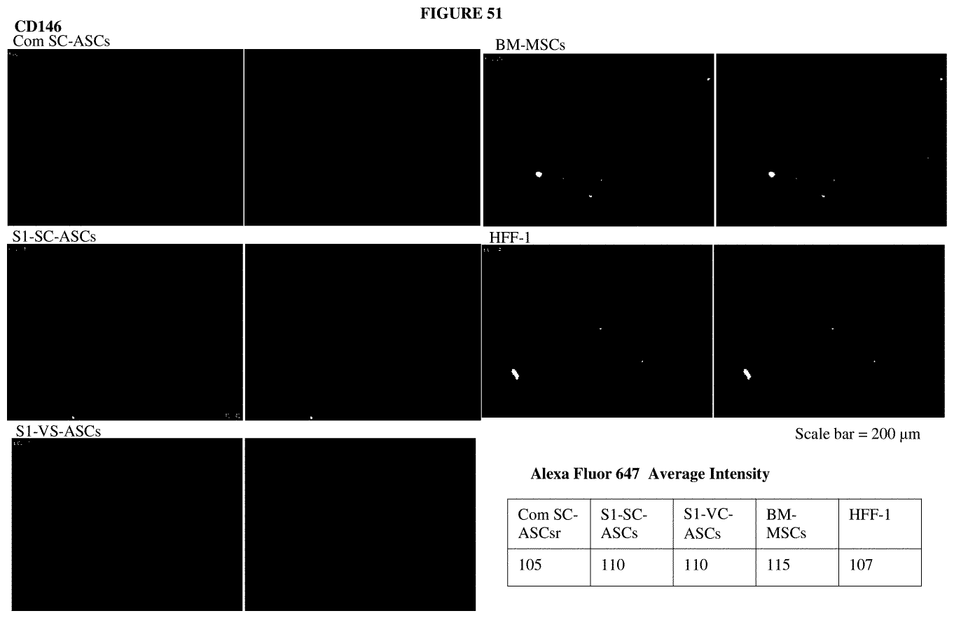

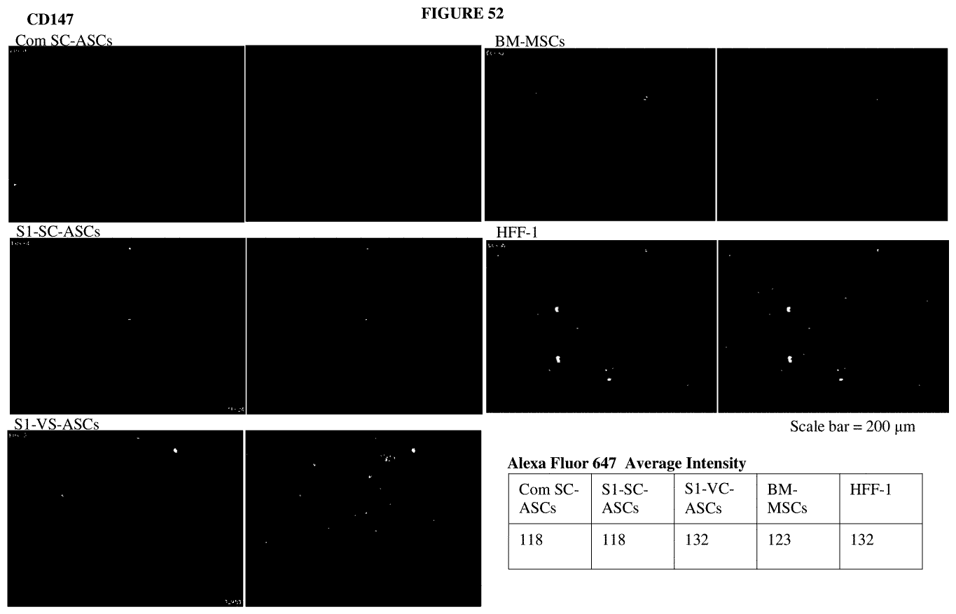

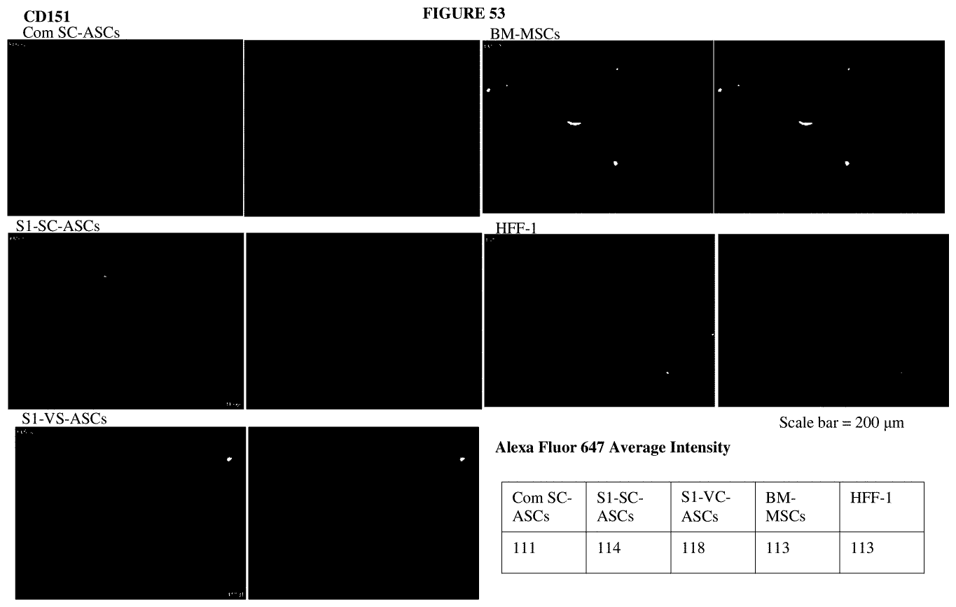

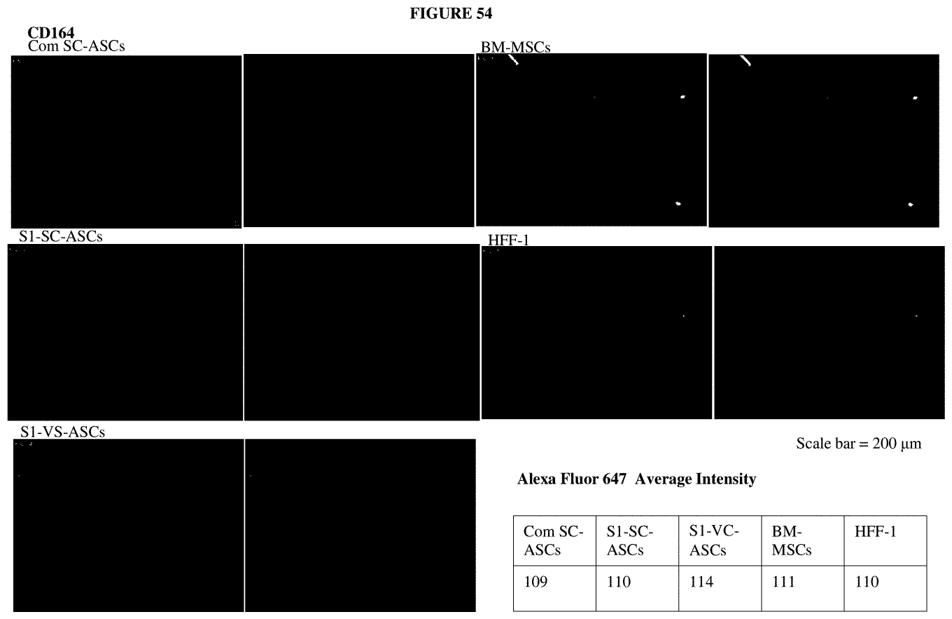

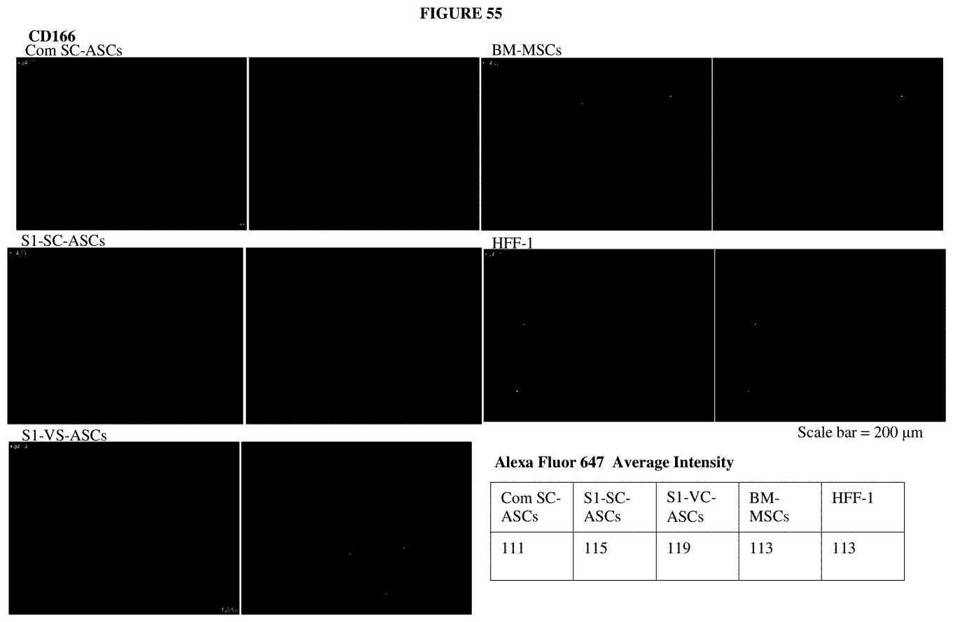

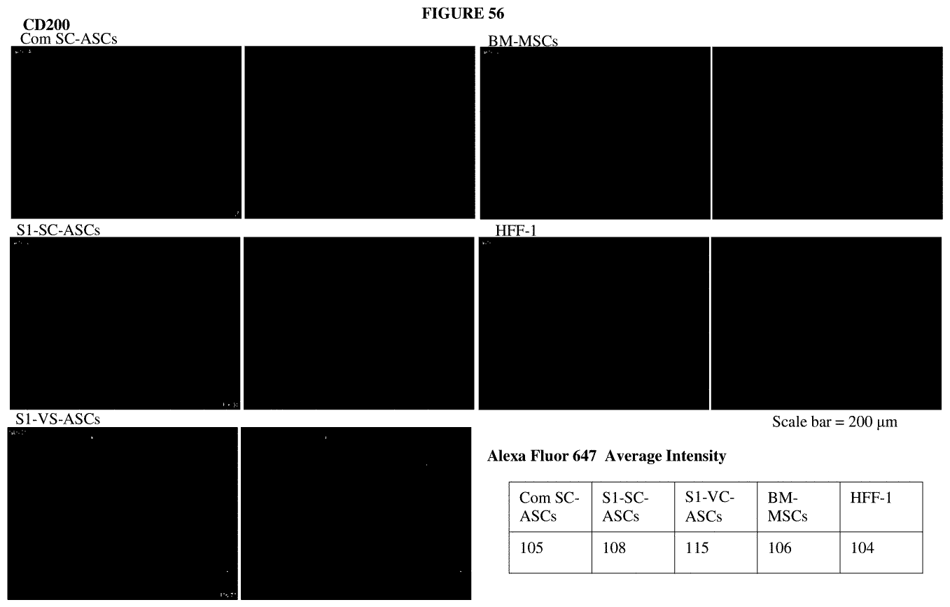

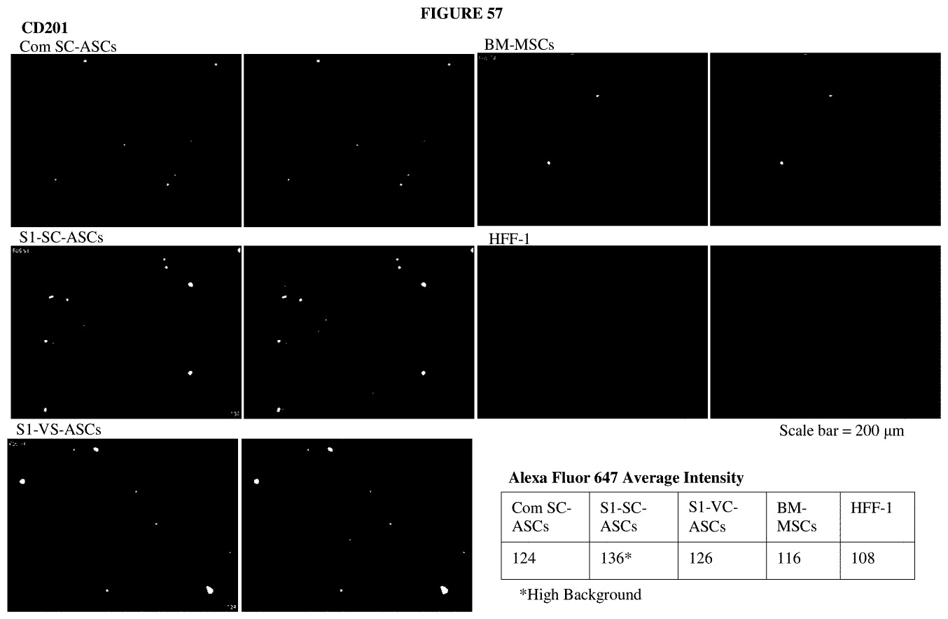

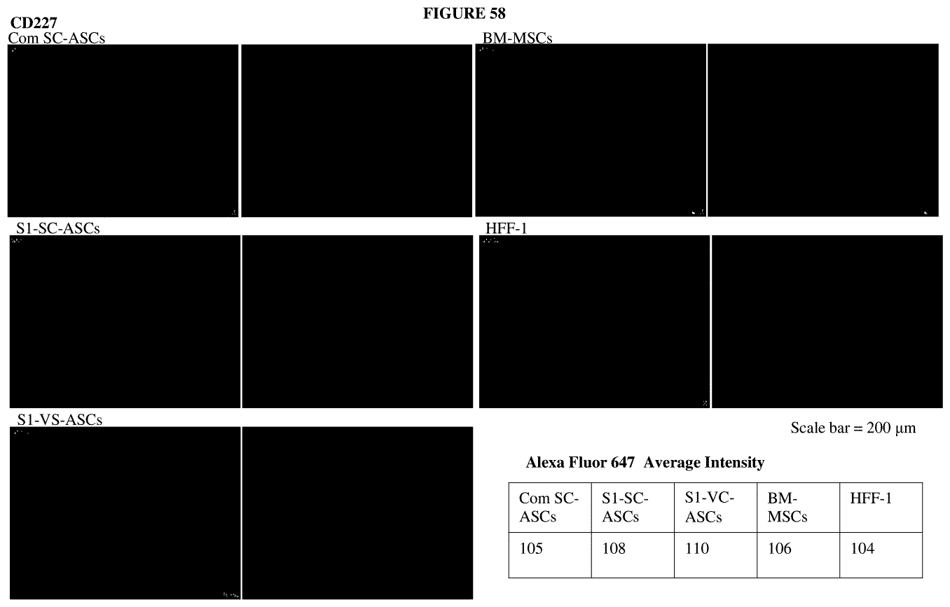

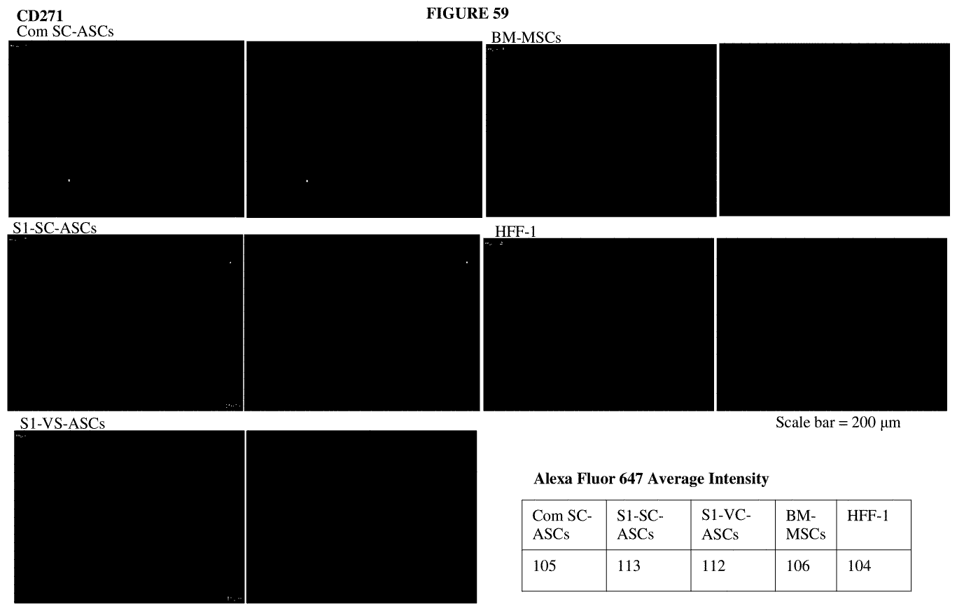

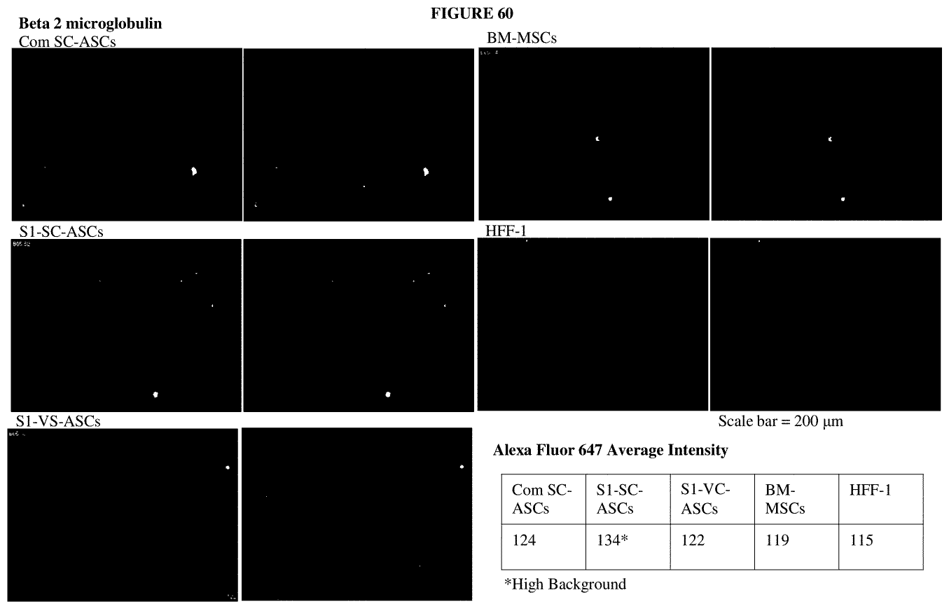

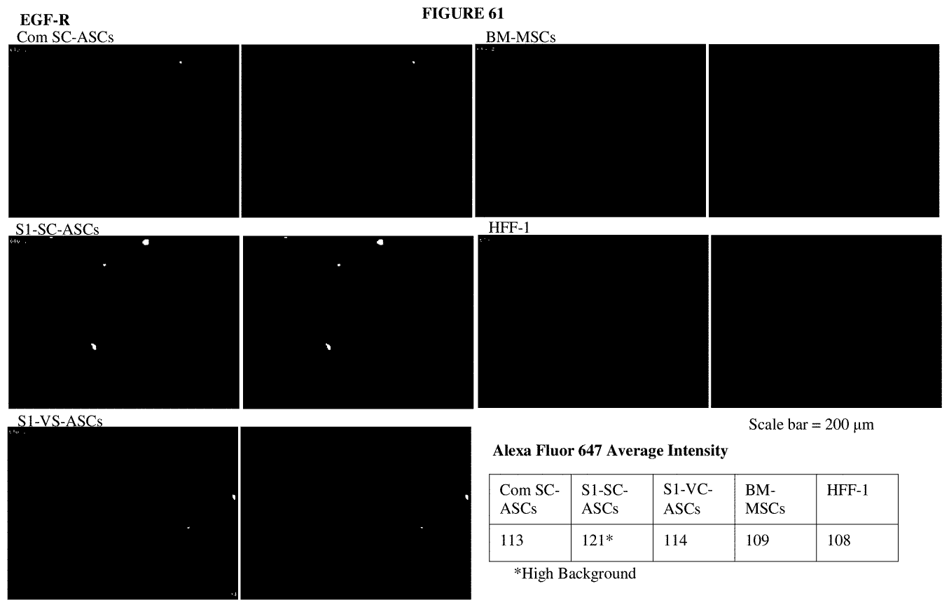

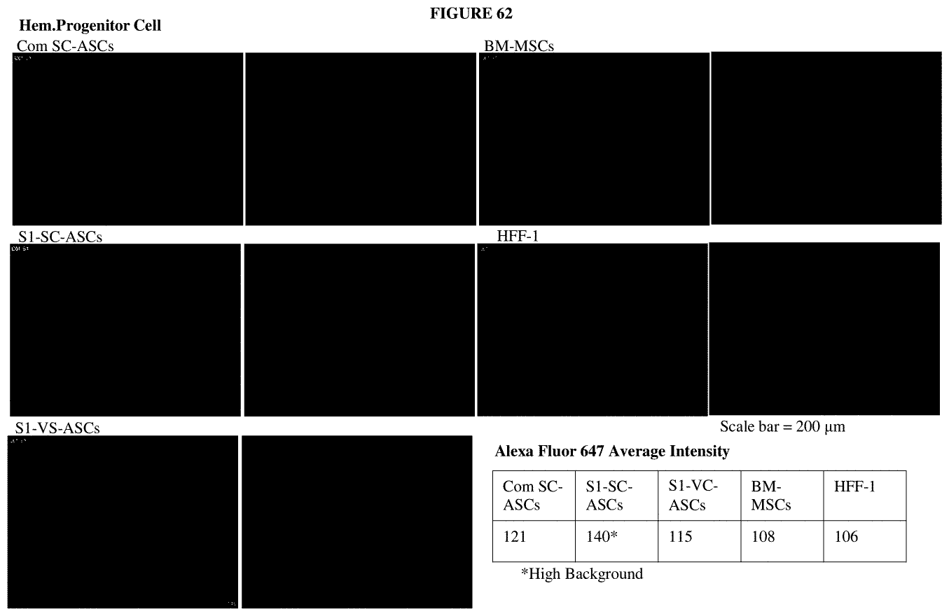

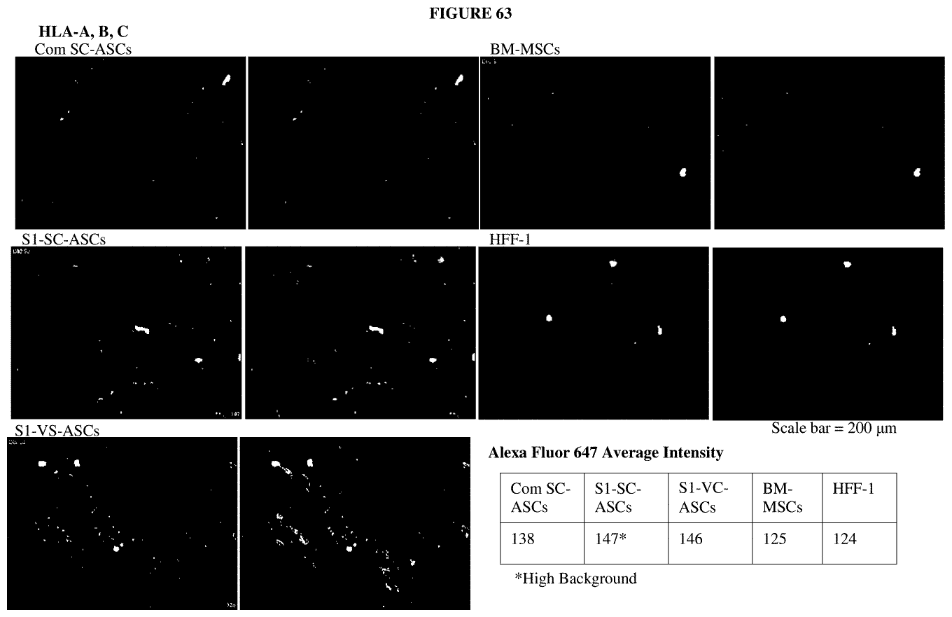

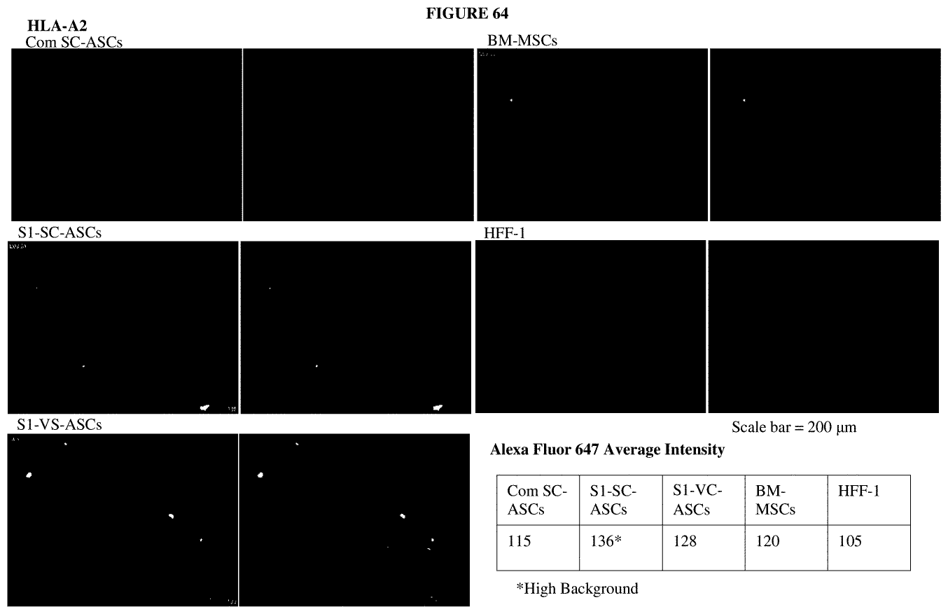

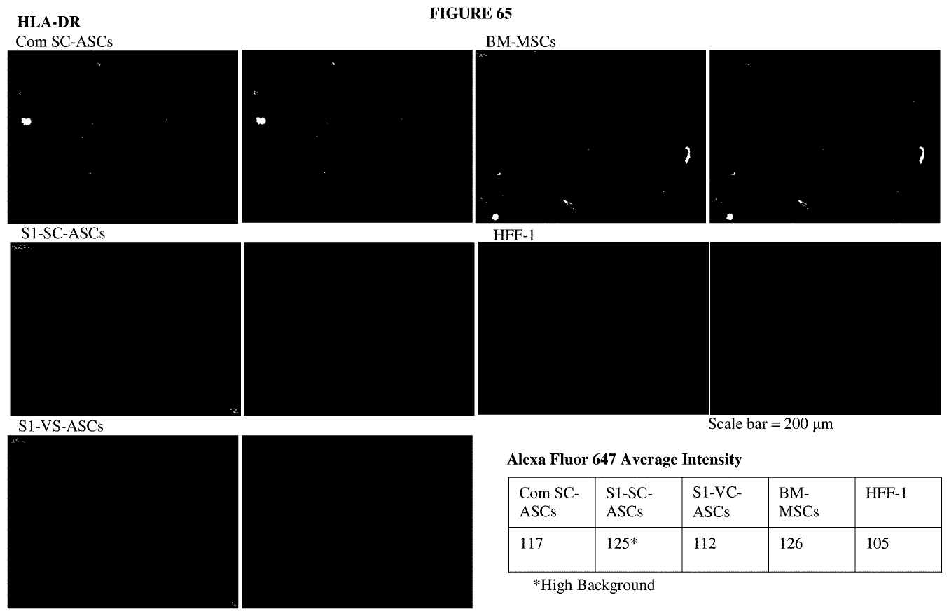

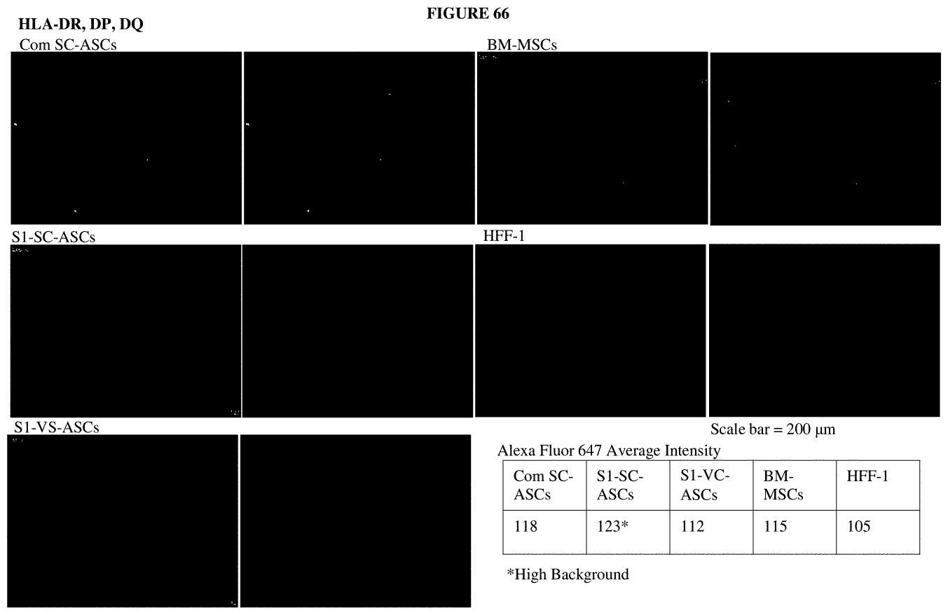

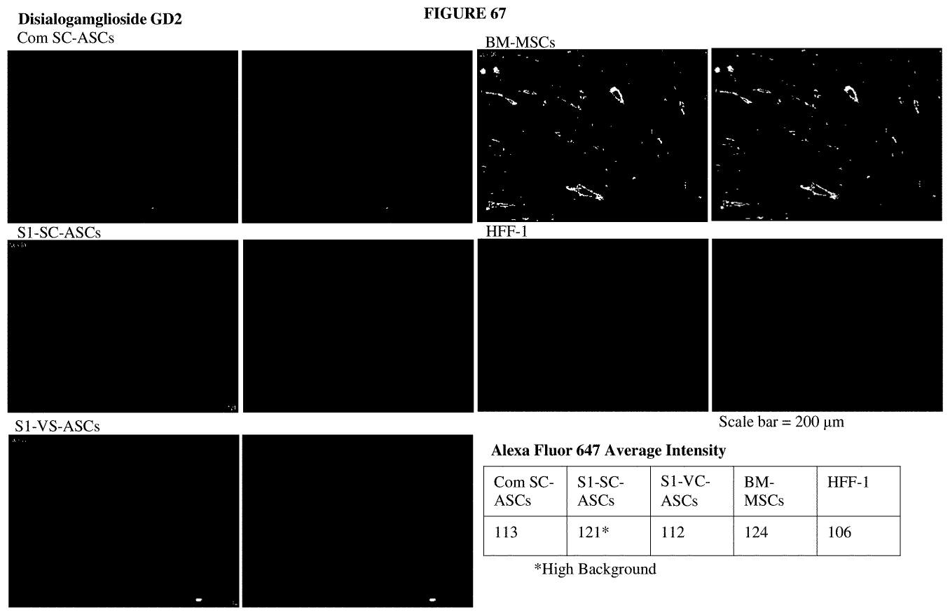

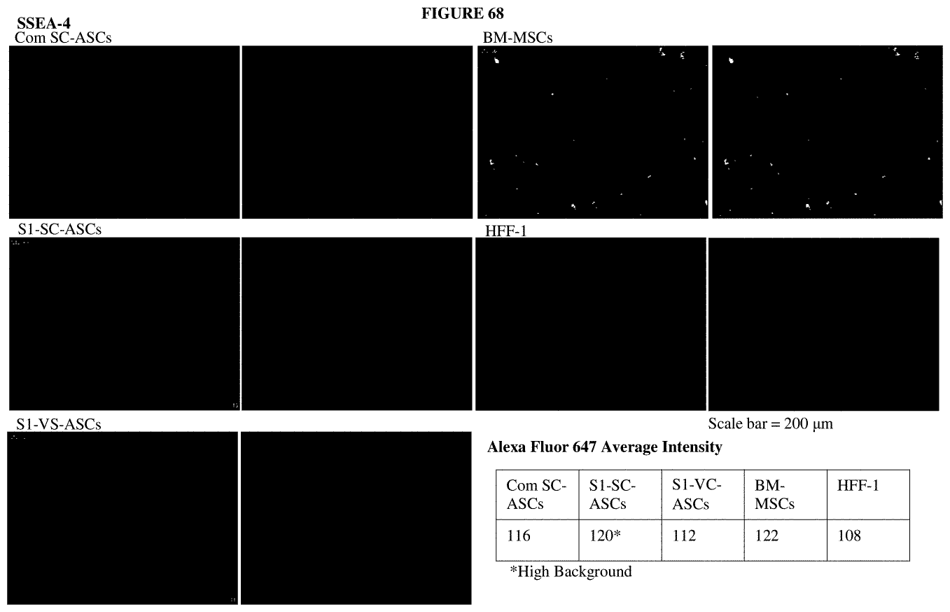

Both subcutaneous and visceral adipose-derived stem cells of subject S1 as well as bone marrow derived-mesenchymal stem cells were positively immunostained with the conventional mesenchymal markers, CD73, CD90 and CD105 (FIG. 1B). The negative control HFF-1 was also stained positively to some extent, especially for CD90, indicating limitation of the existing markers. Based on the fluorescence intensity analysed using image analysis software and visual confirmation, cell surface marker candidates that showed differential immunofluorescence signals between subcutaneous and visceral adipose-derived stem cells in the high content screening were selected for further studies. Fluorescence images of all the markers that had at least one positive signal are shown in FIGS. 11 to 68. Examples of these included CD10 and CD141 as potential subcutaneous adipose-derived stem cells markers, whereas CD142 and CD200 were selected as potential visceral adipose-derived stem cells (FIG. 1C). Without being bound by theory, it was deduced that intrinsic differences in their molecular properties lead to the phenotypic variation of subcutaneous adipose-derived stem cells and visceral adipose-derived stem cells and that that was reflected by the expression difference in their cell surface markers. It was seen that CD10^{hi} cells sorted from subcutaneous adipose-derived stem cells differentiated better than their CD10^{lo} counterparts, whereas CD200^{lo} visceral adipose-derived stem cells differentiated better than CD200^{hi} visceral adipose-derived stem cells. The expression of CD10 and CD200 is thus depot-dependant and associates with adipogenic capacities.

### Example 3: Immunofluorescence Analysis for Marker Expression

Follow-up immunofluorescence studies, immunostaining and immunoblot assays were performed independently to study and verify the expression of selected cell surface marker candidates across subjects S1 to S3, as well as of commercial subcutaneous adipose-derived stem cells, bone marrow-derived mesenchymal stem cells and HFF-1. At the final stage, a total of 12 human subjects and mice, with either normal weight or obesity, were investigated for determination of specific markers by experimental analyses such as flow cytometry and quantitative PCR.

The immunofluorescence cell images of four regions per well were acquired using automated fluorescence microscope system (ImageXpress Micro, Molecular Device). Fluorescence intensity was analysed by using MetaXpress cellular image analysis software and visual confirmation. Marker candidates were selected based on combination of two methods. First, signal intensities estimated by the software are considered. In addition, manual visual identification was performed for those samples with signal intensities are too weak to give significant values due to different efficiencies of staining by individual antibodies.

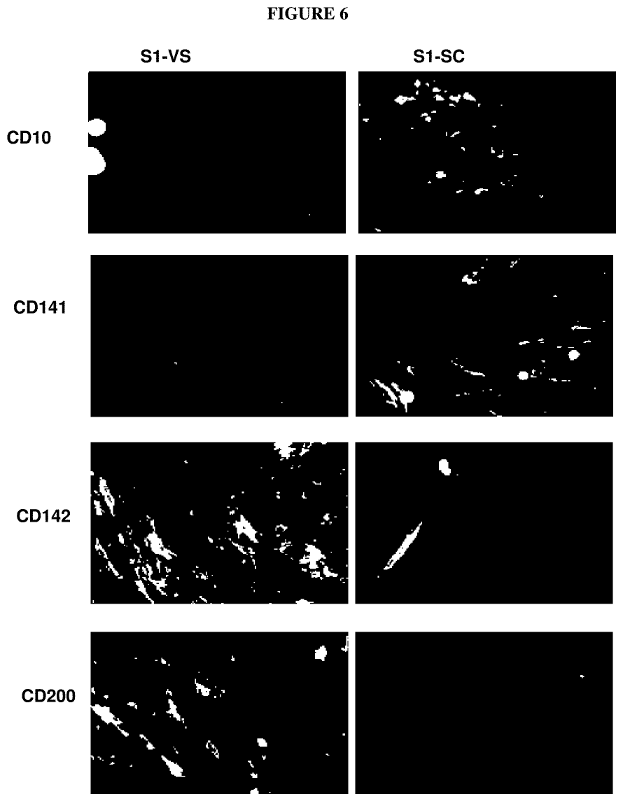

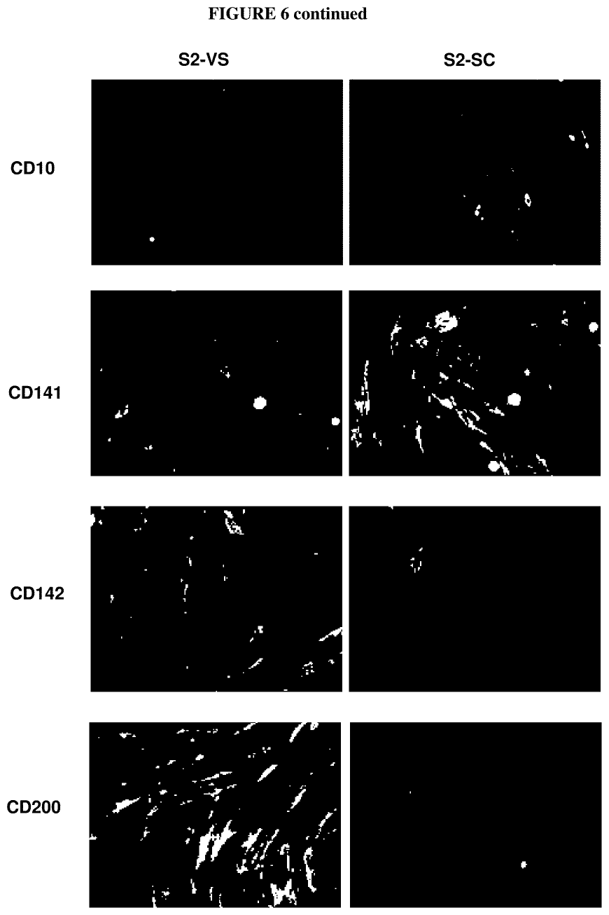

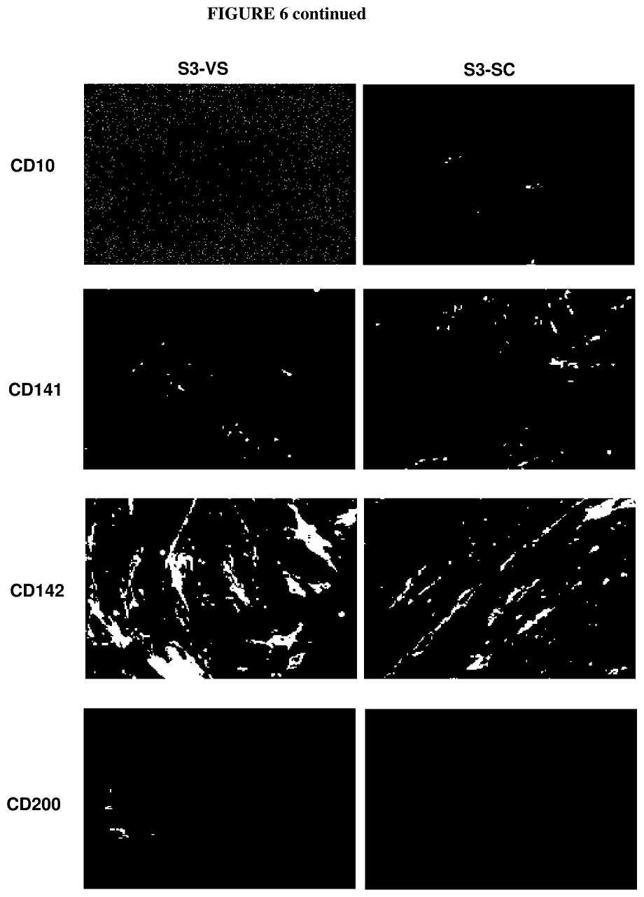

The results demonstrated that predominant expression of CD10 in subcutaneous adipose-derived stem cells and that of CD200 in visceral adipose-derived stem cells was consistent across subjects S1 to S3 (FIG. 6). On the other hand, CD141, for example, was found to be predominantly expressed in subcutaneous adipose-derived stem cells in subject S1, but not in subjects S2 and S3. Similarly, CD142 was predominantly expressed in visceral adipose-derived stem cells in subjects S1 and S2, but not in subject S3 (FIG. 6). Variations were also found in other cell surface markers, consistent with the recent report of heterogeneities in surface marker expression found among individual subcutaneous adipose-derived stem cells. Therefore, heterogeneous markers such as CD141 and CD142 were excluded, and CD10 and CD200 were selected for further studies.

### Example 4: Real Time Quantitative Polymerase Chain Reaction (qPCR)

Total RNA from the cultured cells was extracted using Trizol reagent (Invitrogen) and treated with DNase I to remove genomic DNA. cDNA conversion was performed using the RevertAid H minus first strand cDNA synthesis kit (Fermentas, USA) with oligo d(T) 18 primer according to manufacturer's instructions. For qPCR, cDNA samples were analysed in triplicates using the SYBR® Green PCR Master Mix reagent kit (Applied Biosystems) on a StepOnePlus™ Real-Time PCR System (Applied Biosystems) using the primer pairs shown in Table 2. Relative mRNA levels were calculated and normalized to that of GAPDH.

TABLE 2
Primer pairs for Real time quantitative
PCR analysis

| | SEQ ID | | SEQ ID | |
|---|---|---|---|---|
| Gene | NO: | Forward | NO: | Reverse |
| hCD10 | 1 | TTGTTGGCACTGA | 2 | TCCAGTGCATTCATA |
| | | TGATAAGAATTC | | GTAATCTCTAGAAG |
| hCD200 | 3 | TGTGACCGACTTT | 4 | TTAGGGCTCTCGGTC |
| | | AAGCAAACCGTC | | CTGATTCC |
| mCd10 | 5 | GAACTTTGCCCAG | 6 | GCGGCAATGAAAGG |
| | | GTGTGGT | | CATCTG |
| mCd200 | 7 | GAGCACAGCTCAA | 8 | GTTTTCTGGGCTCAC |
| | | GTGGAAGT | | GGCTT |
| hPPARG | 9 | GACAGGAAAGACA | 10 | GGGGTGATGTGTTTG |
| | | ACAGACAAATC | | AACTTG |
| haP2 | 11 | CCTTTAAAAATACT | 12 | GGACACCCCCATCTA |
| | | GAGATTTCCTTCA | | AGGTT |
| hGAPDH/ | 13 | CAAGGTCATCCATG | 14 | GGCCATCCACAGTCT |
| mGapdh | | ACAACTTTG | | TCTGG |

### Example 5: Immunoblot Analysis

Cell lysates were prepared Nonidet-P40 (NP-40) buffer containing protease inhibitors. Protein quantifications were performed using the Bradford Protein Assay (BioRad). Equal amounts of protein (20 μg) were loaded into 10% SDS-PAGE mini gels and blotted onto a nitrocellulose membrane by using the iBlot Dry Blotting System (Invitrogen). Membranes were blocked in 3% BSA for 1 h at room temperature. They were then probed with primary antibodies overnight at 4° C., followed by HRP-conjugated secondary antibodies for 1 h at room temperature. Primary antibodies used were anti-CD10 (Leica Microsystmes CD10-270-L-CE) and anti-β-Actin (Santa Cruz).

### Example 6: Flow Cytometry and Cell Sorting

For immunophenotypic characterization, ASCs were trypsinised and a total of 2×10⁵ cells were suspended in 0.2 ml staining buffer (DMEM without phenol red with 2% FBS) for immunostaining. The cells were incubated for 30 min on ice, either directly with a fluorochrome-conjugated antibody or with an unconjugated primary antibody followed by an appropriate secondary antibody conjugated with fluorochrome. Antibodies against CD10, CD73, CD90, CD105 and CD200 were of the same clones as those provided in the BD Lyoplate (BD Biosciences, USA). The stained cells were washed and suspended in sorting buffer (PBS with 0.5% BSA and 2 mM EDTA) before analysis by flow cytometry (LSRII, BD Biosciences). Cells for sorting were processed in a similar manner before sorted by Moflo XDP Cell Sorter (Beckman Coulter).

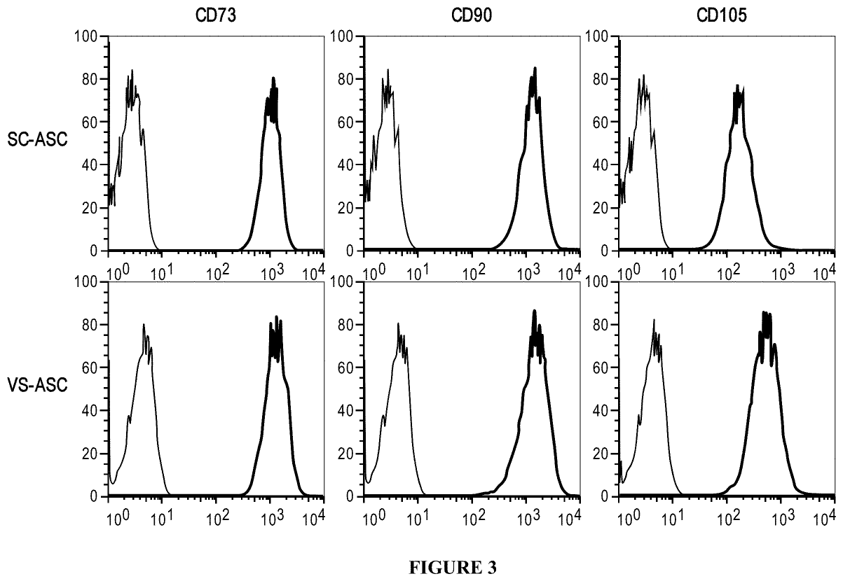

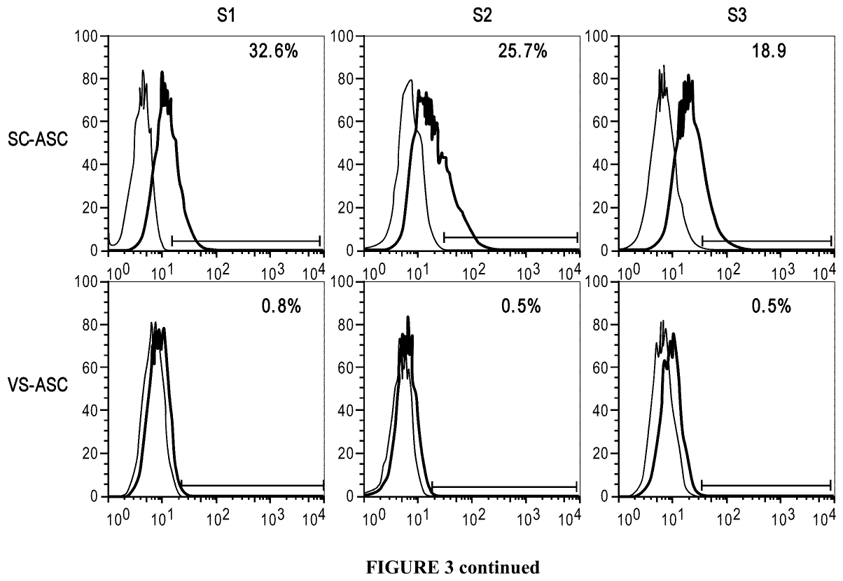

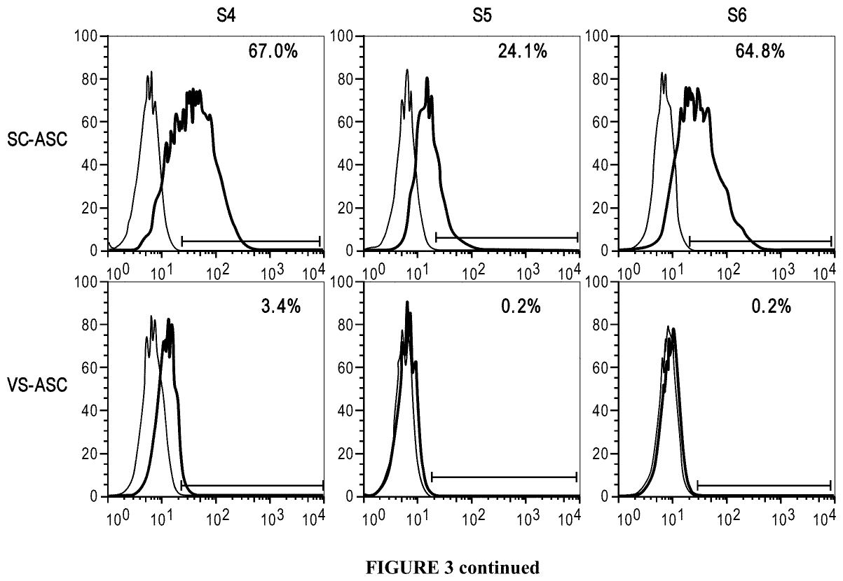

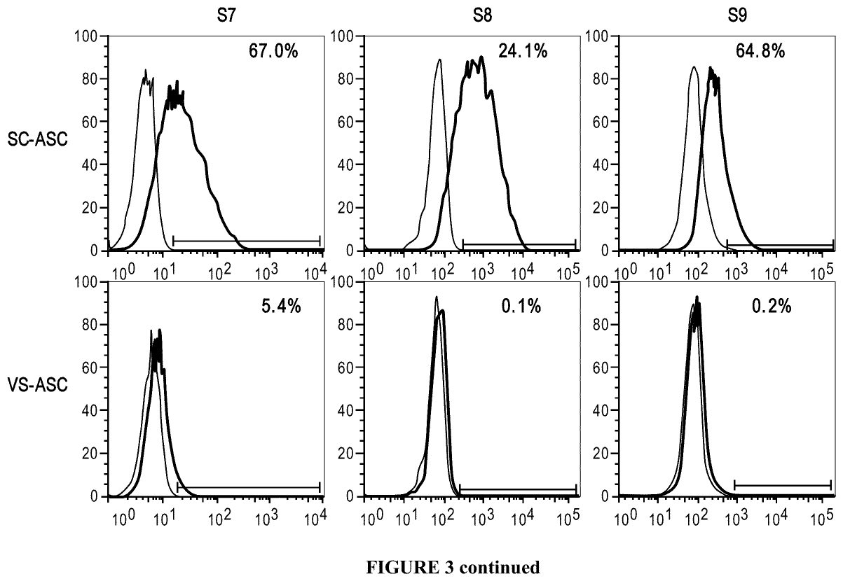

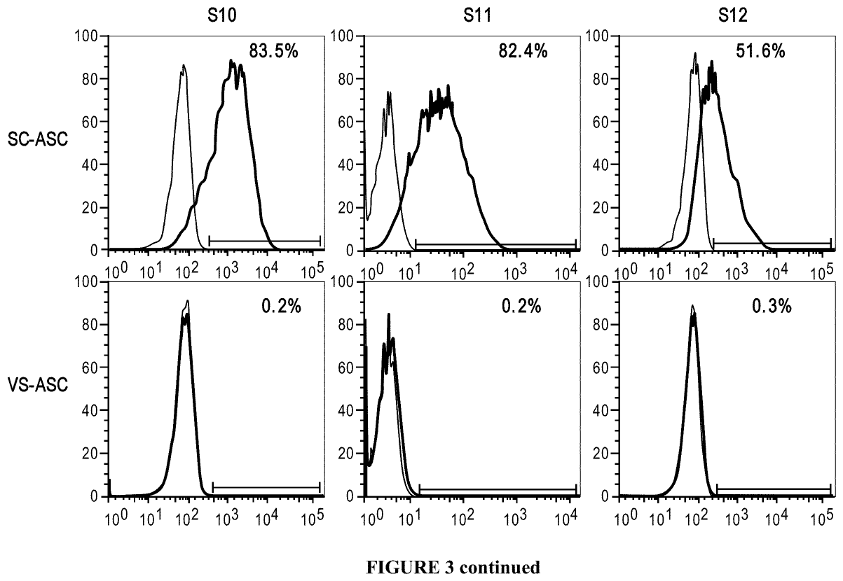

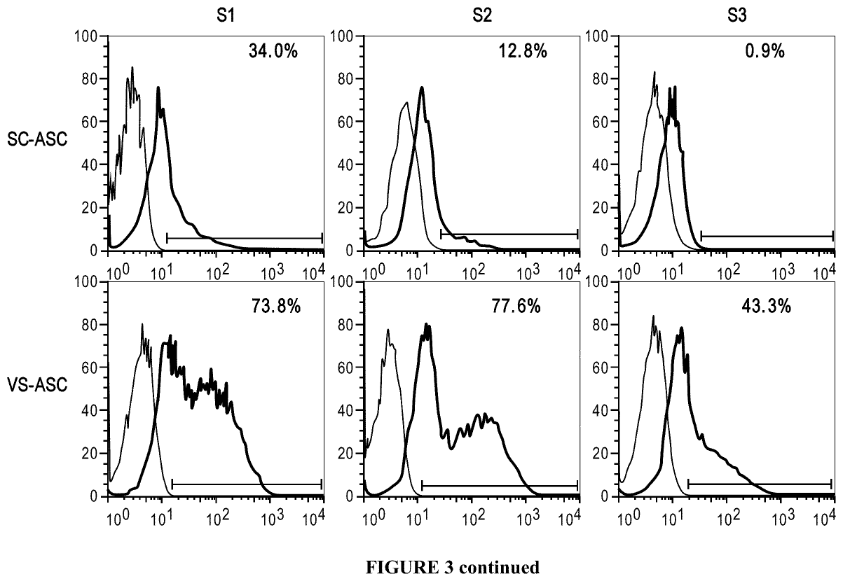

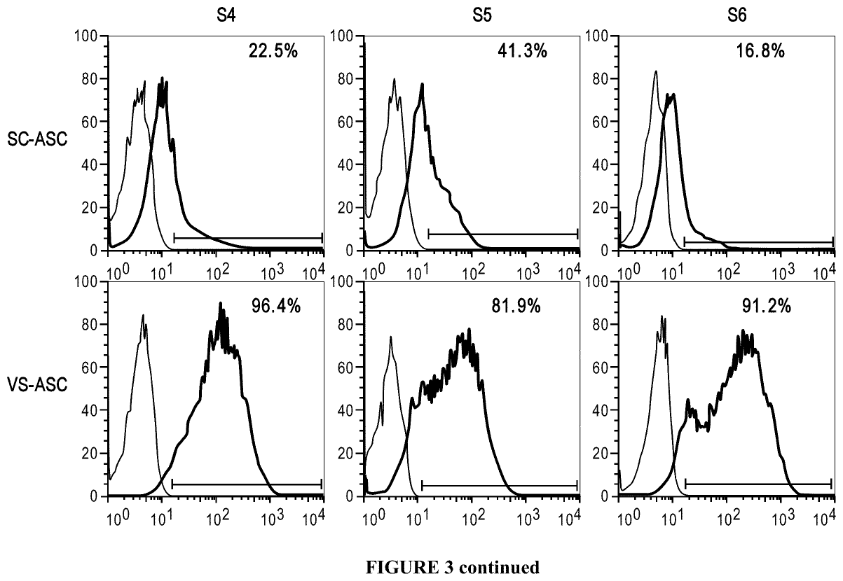

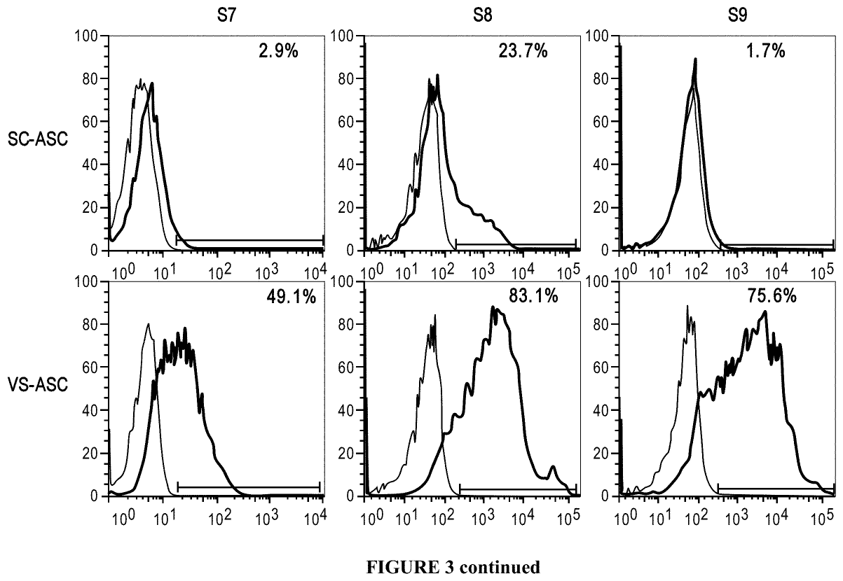

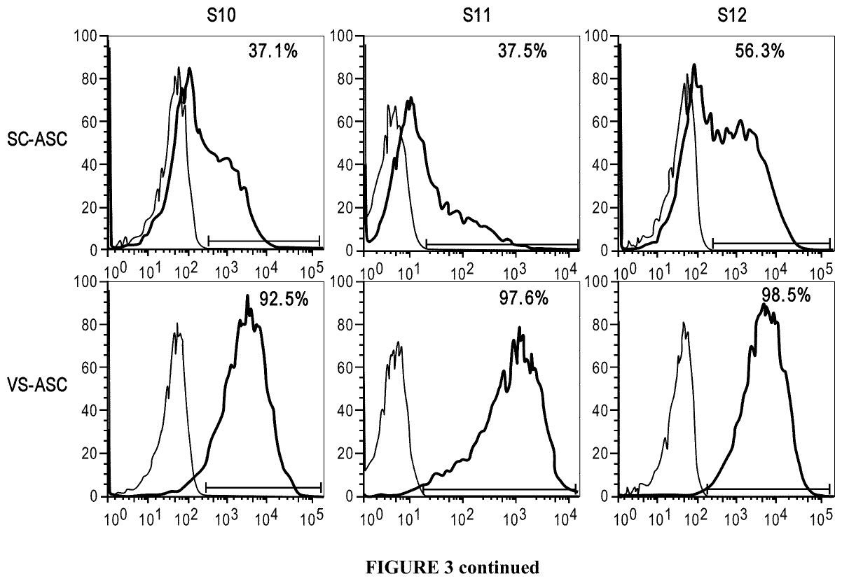

The flow cytometry analysis was performed to study the subpopulation composition of the cultured adipose-derived stem cells. As control, both the subcutaneous and visceral adipose-derived stem cells of all subjects expressed the conventional mesenchymal stem cell markers by this analysis (FIG. 3A). Flow cytometry showed predominant expression of CD10 by subcutaneous adipose-derived stem cells [CD10+ subcutaneous adipose-derived stem cells: mean 40.8% (range of 16.7%-69.1% across subjects S1 to S12) versus CD10+ visceral adipose-derived stem cells: mean 1.9% (0.2%-6.6%)] (FIG. 3B). Similarly, higher expression of CD200 by visceral adipose-derived stem cells [CD200+ subcutaneous adipose-derived stem cells: mean 21.0% (4.3%-40.2%) versus CD200+ visceral adipose-derived stem cells: mean 70.6% (45.9%-95.4%)] was observed (FIG. 3C). These validation result further established CD10 as a subcutaneous-adipose-derived stem cells-specific marker and CD200 as a visceral adipose-derived stem cells-enriched marker.

### Example 7: mRNA and Protein Expression of CD10 and CD200

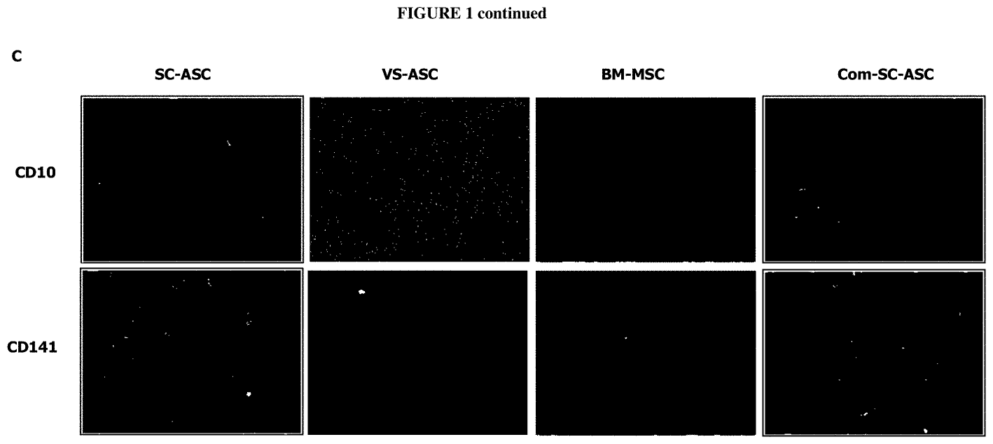

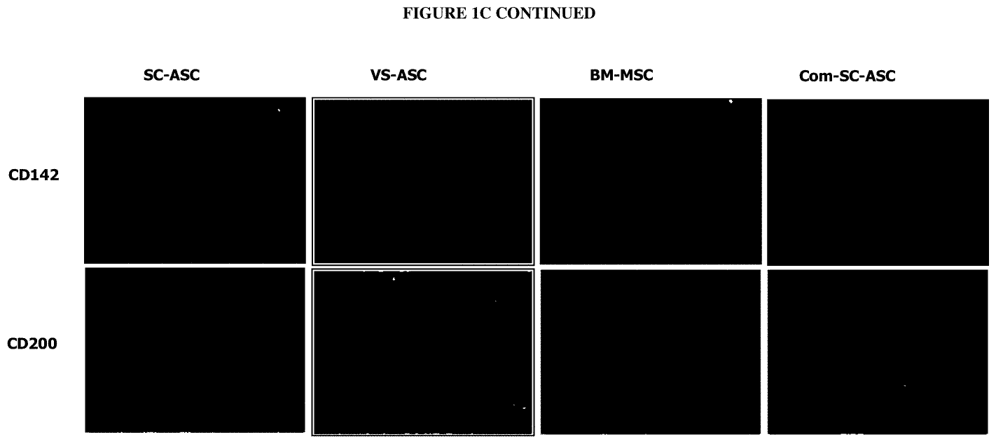

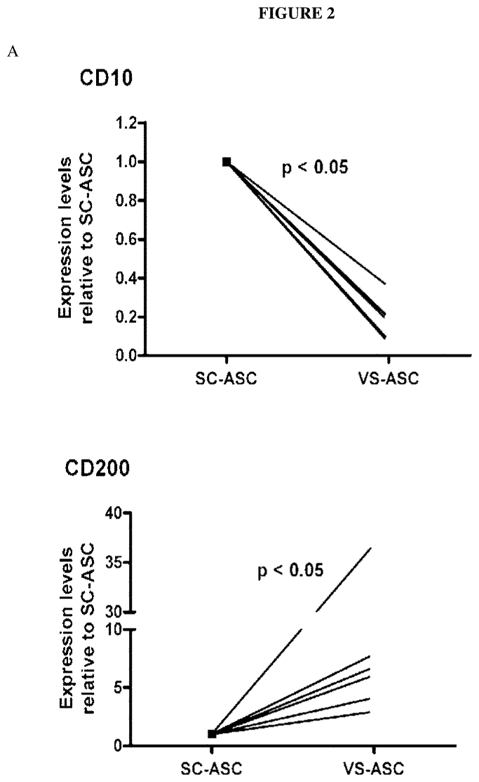

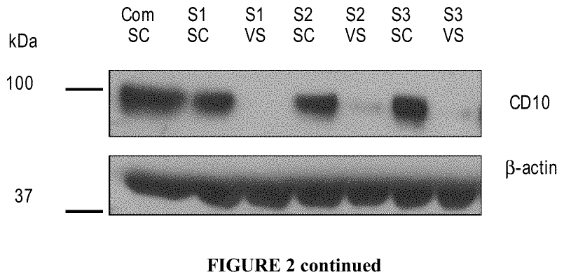

Real-time quantitative polymerase chain reaction (qPCR) showed that the mRNA expression of CD10 was consistently higher in the subcutaneous adipose-derived stem cells relative to visceral adipose-derived stem cells, whereas that of CD200 was consistently lower in the subcutaneous adipose-derived stem cells relative to visceral adipose-derived stem cells across subject S1 to S6 (FIG. 2A). This was complemented by protein expression analysis using Western blotting of CD10 (FIG. 2B).

Results indicate that adipose-derived stem cells from visceral depots would not express CD10, in contrast to commonly studied adipose-derived stem cells from subcutaneous depots. A high CD200 expression in mesenchymal stem cells isolated from Wharton's jelly was shown, compared to bone-marrow-derived mesenchymal stem cells and (presumably subcutaneous) adipose-derived stem cells. It was hypothesized that the inflammatory environment of Wharton's jelly contributed to higher mesenchymal stem cell expression of CD200. Visceral fat depots are thought to have similar inflammatory conditions and may result in abundant expression of CD200. Furthermore, CD200 was identified as a marker to enrich endocrine cell populations derived from human embryonic stem cells in order to isolate pancreatic progenitor lineages, possibly indicating an endocrine origin of visceral fat-derived cells.

### Example 8: Mouse Studies

39 week old male C57BL6/J mice were fed either with normal chow or high fat diet for 27 weeks (5 mice per feeding group). Inguinal (subcutaneous), epididymal and mesenteric fat depots were harvested, and ASCs were isolated as described herein. mRNA was isolated from the cultured ASCs, then used for the qPCR analysis. This animal work was approved by the Institutional Animal Care and Use Committee of Biological Resource

Centre, Singapore.

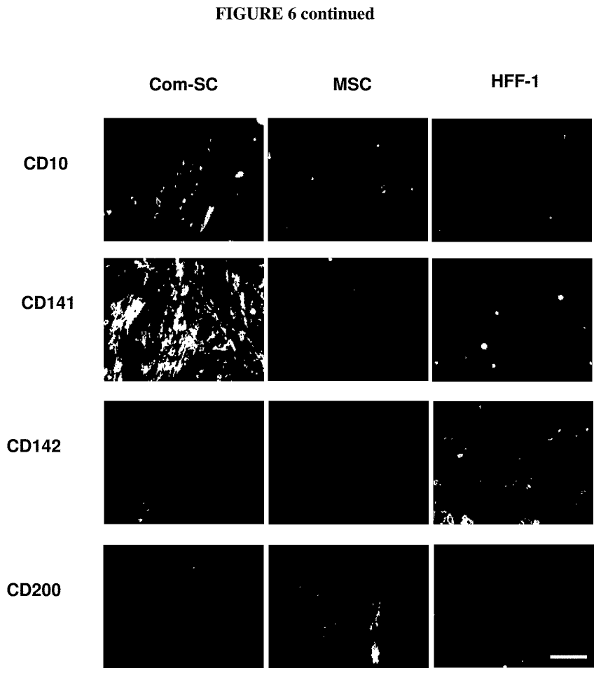

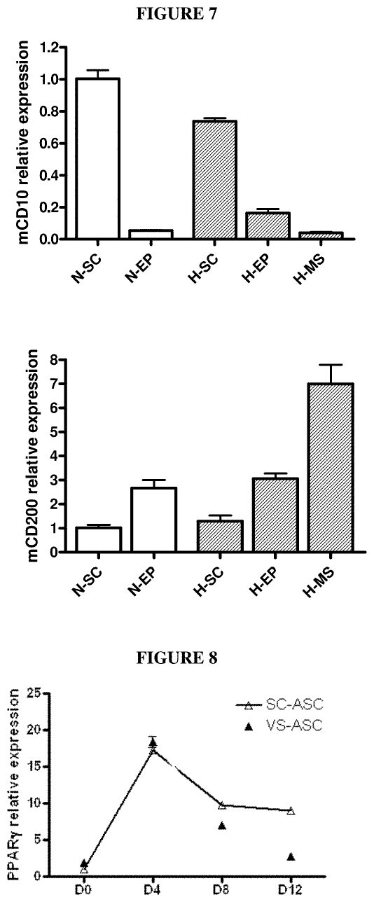

In order to verify that these depot-specific markers are also valid in another species, the expression of CD10 and CD200 was investigated in mouse. C57BL6/J mice were either fed normal chow (NC), or high fat diet (HFD) to become obese. The body weight was 39.9±0.83 g for NC and 60.0±0.95 g for HFD mice (n=5). Mouse adipose-derived stem cells from inguinal (subcutaneous), epididymal (visceral) and mesenteric (visceral) fat depots were isolated from these animals. The qPCR analysis indicated that CD10 is predominantly expressed in subcutaneous fat, while CD200 is highly expressed in visceral (epididymal and mesenteric) fat in both NC- and HFD-fed mice (FIG. 7). This result suggests that the depot specificity of CD10 and CD200 holds true in mouse species and that the diet condition would not affect this specificity.

### Example 9: Oil Red O Staining

For detecting lipids in cells and to ascertain the adipogenic capability of the same, cells were seeded in 12- or 24-well plates and adipogenesis was induced on day 0, 2 days after the cells reaching confluence state, with adipogenic cocktail containing 1 μM dexamethasone, 0.5 mM isobutylmethylxanthine (IBMX) and 167 nM insulin plus 1 μM pioglitazone or 100 μM indomethacin in the absence of bFGF. On day 4, cells were switched to medium with 167 nM insulin and maintained till at least day 12. The cells were then fixed in 3.7% formaldehyde in PBS for 1 h, washed with 60% isopropanol, air-dried and stained with Oil Red 0 solution (in 60% isopropanol) for 1 h followed by repeated washing with water. Stained cells were imaged on Nikon TS100 microscopes. After air-dried, stained lipids were extracted by using isopropanol for lipid content measurements. The absorbance of extracted solution was measured at 500 nm wavelength.

### Example 10: Expression Level of CD10 and CD200 During Adipogenesis and the Adipogenic Potential of Adipose-Derived Stem Cell Subpopulations Expressing High/Low Level of CD10 or CD200

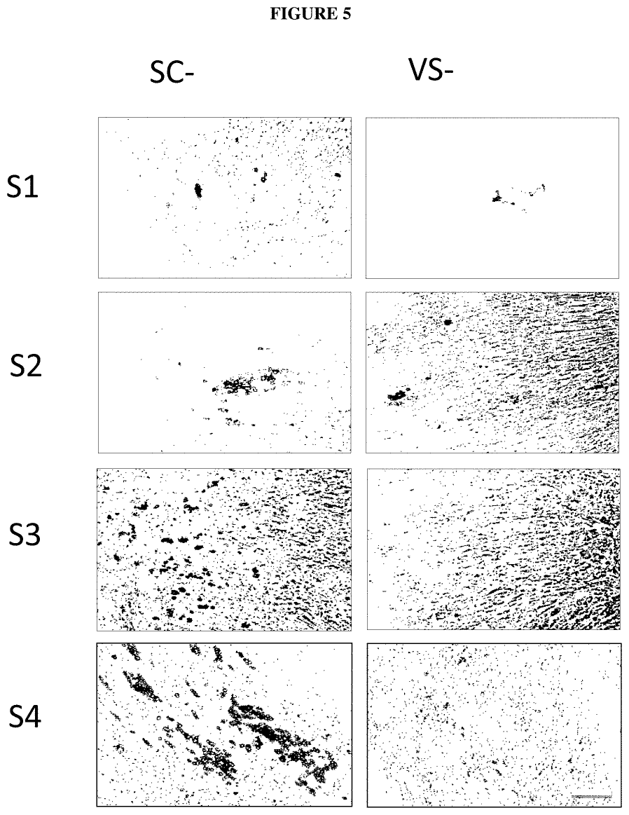

Subcutaneous and visceral adipose-derived stem cells were subjected to in vitro standard adipocytic differentiation cocktail (e.g., insulin, dexamethasone and IBMX). It was seen that subcutaneous adipose-derived stem cells underwent robust adipogenesis, whereas visceral adipose-derived stem cells are relatively resistant to the adipogenic stimuli as revealed by Oil Red 0 staining (FIG. 5). This trend of adipogenic-prone subcutaneous adipose-derived stem cells and adipogenic-resistant visceral adipose-derived stem cells persisted at all the time points even when the standard adipogenesis cocktail was augmented with pioglitazone or indomethacin, strong inducers of adipogenesis by potently activating PPARγ, a master regulator of adipogenesis.

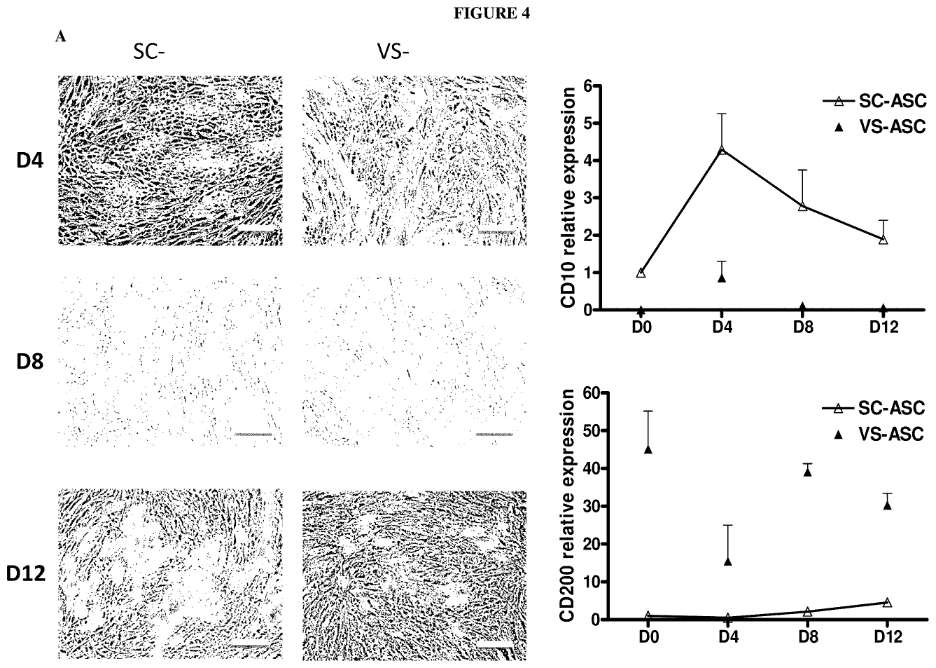

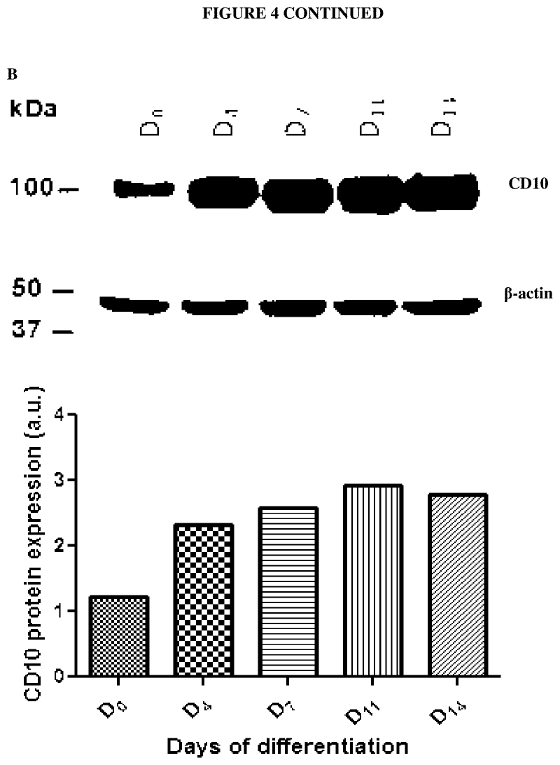

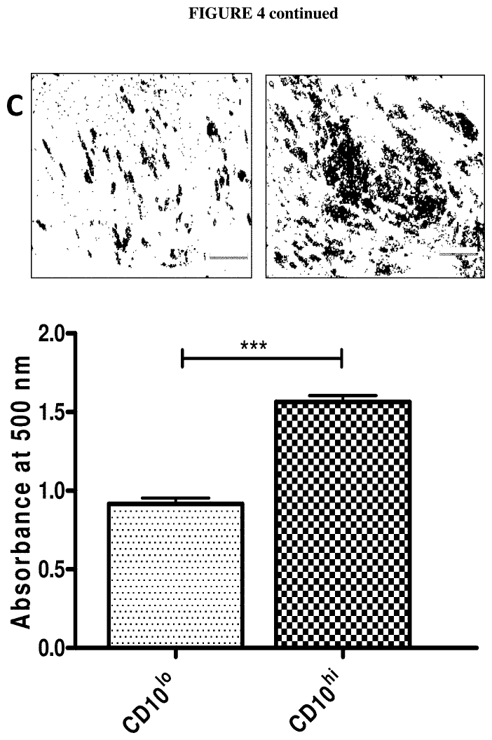

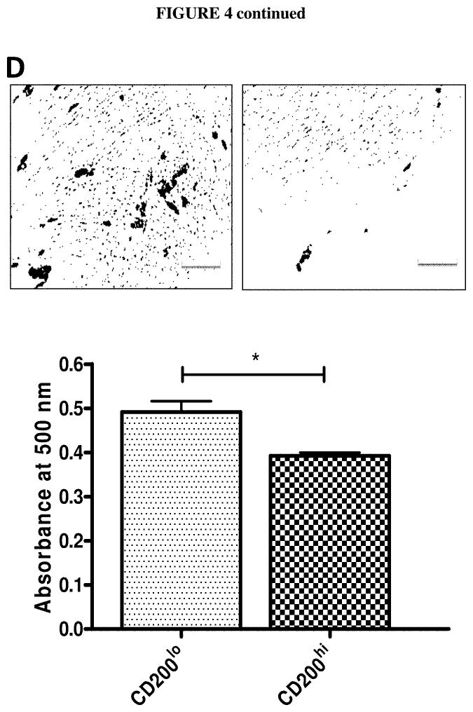

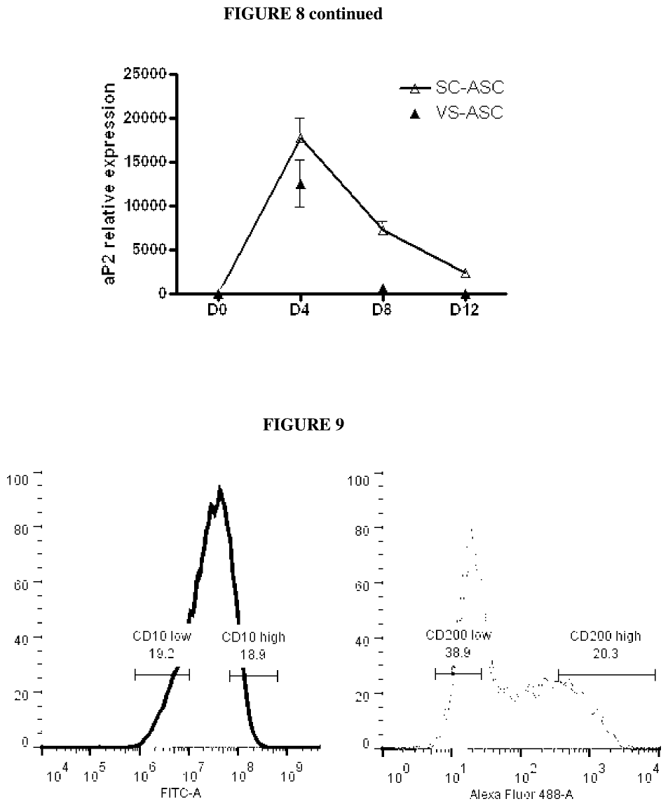

As described herein, subcutaneous adipose-derived stem cells differentiate better into mature adipocytes than visceral adipose-derived stem cells using the standard in vitro adipogenesis protocol. In order to investigate the changes in expression of CD10 and CD200 during adipogenesis, qPCR was performed in differentiating subcutaneous adipose-derived stem cells and visceral adipose-derived stem cells. The mRNA expression level of CD10 increased after adipogenic stimuli and this increase positively correlated with those of adipogenic markers, PPARγ and aP2 (FIGS. 4A and 8). In contrast, the CD200 level decreased after adipogenesis is initiated, and a negative correlation with those of PPARγ and aP2 was observed (FIG. 4A). Increase of CD10 during adipogenesis of subcutaneous adipose-derived stem cells was also confirmed at the protein level by Western blot (FIG. 4B).

The results herein led to the postulation that subcutaneous adipose-derived stem cells specific CD10 may mark cell populations with more adipogenic capacities, whereas visceral adipose-derived stem cells specific CD200 may mark those with less adipogenic capacities, which are consistent with intrinsic properties of respective adipose-derive stem cells. In order to test the hypothesis, subcutaneous adipose-derived stem cells were sorted into two populations: CD10^{hi} and CD10^{lo} cells (Figure S5A). Similarly, visceral adipose-derived stem cells were sorted into CD200^{hi} and CD200^{lo} cells (Figure S5B). These populations were subjected to the standard in vitro adipogenesis cocktail. The result indicated that CD10^{hi} cells sorted from subcutaneous adipose-derived stem cells differentiate significantly better than their CD10^{lo} counterparts, as revealed by oil red 0 staining and absorbance reading of its extracted solvents (FIG. 4C). Conversely, CD200^{lo} cells sorted from visceral adipose-derived stem cells were found to differentiate significantly better than the CD200^{hi} counterparts (FIG. 4D). These data suggest that CD10 is a prospective marker for high adipogenic potentials, whereas CD200 is a predictive marker for lower adipogenic capacities.

It has been demonstrated herein that CD10 and CD200 show consistent differential expression profiles between subcutaneous and visceral adipose-derived stem cells. The expression level of these markers correlated with the cell's adipogenic capacity, that is, there is a positive correlation of CD10 and a negative correlation of CD200 with probability of adipogenesis. Thus, CD10 and CD200 can be used as prospective markers for the identification of the adipogenic capability of adipose-derived stem cells. Despite importance of cell surface markers to identify and track stem cells, including mesenchymal stem cells and adipose-derived stem cells and known pathophysiological differences between subcutaneous and visceral fat depots, previous studies that have comprehensively examined differences of surface marker expression in the depot-specific manner have not been found. Identification of these depot-specific markers would allow differential isolation, visualisation and characterisation of adipose-derived stem cells in the depot-specific manner.

1-20. (canceled)

21. A method of detecting at least one cell-surface marker selected from the group consisting of CD10, CD141, CD142 and CD200, wherein the cell surface markers are used in a high-throughput screening assay for drugs or compounds that would enhance adipogenesis.

22. The method of claim 21, wherein the compounds convert cells with dysfunctional adipogenic capacity into cells with functional adipogenic capacity.

23. The method of claim 21, wherein the method comprises detecting CD10 and CD141.

24. The method of claim 22, wherein the method comprises detecting CD10 and CD141.
